SEQUENCE LISTING (2 sequences)

SEQ ID NO: 1 - protein, 428 aa, Bunyaviridae species--hantavirus
MSTLKEVQDNITLHEQQLVTARQKLKDAERAVELDPDDVNKSTLQSRRAAVSALETKLGELKRELADLIAAQKLASKPVDPTGIEPDDHLKEKSSLRYGNVLDVNSIDLEEPSGQTADWKSIGLYILSFALPIILKALYMLSTRGRQTIKENKGTRIRFKDDSSYEEVNGIRKPRHLYVSMPTAQSTMKADEITPGRFRTIACGLFPAQVKARNIISPVMGVIGFSFFVKDWMERIDDFLAARCPFLPEQKDPRDAALATNRAYFITRQLQVDESKVSDIEDLIADARAESATIFADIATPHSVWVFACAPDRCPPTALYVAGMPELGAFFAILQDMRNTIMASKSVGTSEEKLKKKSAFYQSYLRRTQSMGIQLDQKIIILYMSHWGREAVNHFHLGDDMDPELRELAQTLVDIKVREISNQEPLKL

SEQ ID NO: 2 - protein, 174 aa, Bunyaviridae species--hantavirus
MSTLKEVQDNITLHEQQLVTARQKLKDAERAVELDPDDVNKSTLQSRRAAVSALETKLGELKRELADLIAAQKLASKPVDPTGIEPDDHLKEKSSLRYGNVLDVNSIDLEEPSGQTADWKSIGLYILSFALPIILKALYMLSTRGRQTIKENKGTRIRFKDDSSYEEVNGIRKP

(19) United States
(12) Patent Application Publication
(10) Pub. No.: US 2011/0189730 A1
(43) Pub. Date: Aug. 4, 2011

(54) VIRAL NUCLEOCAPSID PROTEIN AS A MULTIFUNCTIONAL TRANSLATION INITIATION FACTOR AND INCREASED PROTEIN AND POLYPEPTIDE PRODUCTION USING SAME

(76) Inventors: Antonito T. Panganiban, Placitas, NM (US); Mohammed A. Mir, Lenexa, KS (US)

(21) Appl. No.: 13/002,677

(22) PCT Filed: Jul. 17, 2009
(86) PCT No.: PCT/US09/04150

§ 371 Date: Apr. 11, 2011

Related U.S. Application Data
(60) Provisional application No. 61/135,275, filed on Jul. 18, 2008.
(60) Provisional application No. 61/211,481, filed on Mar. 30, 2009.

(51) Int. Cl. C12P 21/00; C12N 15/86; C12N 5/10

(52) U.S. Cl. 435/69.1; 435/456; 435/320.1; 435/325

(57) ABSTRACT

The present invention is directed to a system to significantly increase the expression of genes of interest, and in particular proteins and polypeptide products. Expression of hantavirus nucleocapsid protein (N) by itself results in augmented translational expression of diverse genes. The mechanism of this augmentation relies on the ability of N to replace the cellular cap binding complex to attain more efficient translation initiation—the result being great mRNA production and greater protein/polypeptide production. The inventors have also recently found that inclusion of a 5′ untranslated leader region (viral UTR) from a viral RNA, in conjunction with N, leads to even more robust expression. This mechanism appears to involve recognition of the viral UTR by the N to provide even more robust protein production. Thus, a general strategy for expression of any gene would be to generate significant quantities of mRNA containing the viral UTR from a strong promoter, and then to allow translation of mRNA of a gene product in the presence of N. Even a modest increase in the production of commercially desirable proteins is a goal in industry.

This application claims priority from U.S. Provisional Application Nos. 61/135,275, entitled “Viral nucleocapsid protein as a multifunctional translation initiation factor”, filed Jul. 18, 2008, and 61/211,481, entitled “Increased protein production by bunyavirus nucleocapsid protein and viral 5′ untranslated regions: a translational enhancer-promoter system, filed Mar. 30, 2009, the entire contents of which applications are hereby incorporated by reference herein.

### RELATED APPLICATIONS AND GOVERNMENT SUPPORT

The present invention was made with government support under Grant No. 5R21AI059330 and R01AI074011 awarded by NIH/NIAID. Consequently, the government retains certain rights in this invention.

### FIELD OF THE INVENTION

The present invention relates to eukaryotic mRNA translation, more specifically to eukaryotic mRNA translation wherein the nucleocapsid protein of hantaviruses functions as an initiation factor in the translation of mRNA.

The present invention also relates to nucleotide constructs and processes that significantly increase the expression of genes of interest, thereby yielding increased production of proteins and polypeptides.

### BACKGROUND OF THE INVENTION

Complete citations for the references cited herein are listed in the “References (First Set)” and “References (Second Set)” compilations provided in the “Detailed Description of the Invention”.

Members of the hantavirus genus of the family Bunyaviridae are enveloped viruses harbouring three negative-sense, single-stranded genomic RNA molecules (Schmaljohn, 1996). The nucleocapsid peptide (N) has a vital function in hantavirus replication. Multiple studies show that N recognizes viral RNA (vRNA) with specificity indicative of its function during encapsidation (Gott et al, 1993; Severson et al, 1999, 2001; Osborne and Elliott, 2000; Jonsson et al, 2001; Jonsson and Schmaljohn, 2001; Mir and Panganiban, 2006). Each of the three genome segments form pseudocircular structures through a short imperfect ‘panhandle’ composed of hydrogen-bonded nucleotides from the 5′ and 30 termini (Pettersson and von Bonsdorff, 1975; Obijeski et al, 1976; Raju and Kolakofsky, 1989). The terminal panhandle is both necessary and sufficient for high-affinity binding by N (Mir and Panganiban, 2004b, 2005). N also functions in viral genome replication, as complementary in vitro and in vivo studies indicate that N, from diverse negative-sense RNA viruses, serves in vRNA replication working in coordinated manner with the viral polymerase or through interaction with template RNA (Bridgen and Elliott, 1996; Blakqori et al, 2003; Pinschewer et al, 2003; Kohl et al, 2004; Ikegami et al, 2005). Although hantavirus replication is exclusively cytoplasmic, generation of viral mRNA uses an orthomyxovirus-like capsnatching mechanism yielding mRNAs with 5′ m7G caps derived from cellular mRNAs (Dunn et al, 1995; Garcin et al, 1995; Hutchinson et al, 1996).

The vast majority of eukaryotic mRNA translation is m7G cap dependent. Translation initiation involves the recognition of capped mRNA by a set of initiation factors. (components of eIF4F cap-binding complex) (Dever, 1999; Gingras et al, 1999; Richter and Sonenberg, 2005). This heterotrimeric complex includes eIF4E, which directly binds to the mRNA cap (von der Haar et al, 2004; Richter and Sonenberg, 2005), and eIF4A, which is a DEAD box RNA helicase (Rogers et al, 2002; Hernandez and Vazquez-Pianzola, 2005). The third component of the eIF4 complex is eIF4G, a peptide that interacts with both eIF4E and eIF4A (Mader et al, 1995; Hentze, 1997; Dever, 1999). In addition, eIF4G interacts with eIF3 (Hinnebusch, 2006) to bridge the mRNA-eIF4 cap-binding complex and the 43S ‘pre-initiation complex.’ The 43S complex is composed of the 40S small ribosomal subunit, initiator methionine transfer RNA, eIF2 and GTP (Hershey and Merrick, 2000). Scanning by this large set of proteins then proceeds from the capped 5′ end of the mRNA in a process that may require the helicase activity of eIF4A (Rogers et al, 2002; Hernandez and Vazquez-Pianzola, 2005). When an AUG start codon in optimal context is encountered the 60S large ribosomal subunit and additional factors are recruited and translation begins (Kozak, 1991, 1992).

Typically, viruses use the above-described cellular machinery for translation of their mRNAs, and most have capped mRNAs. However, the picornaviruses, some flaviviruses, a few additional viruses contain a cis-acting internal ribosomal entry sites (IRESs) to enable cap-independent ribosomal entry at a site in the mRNA immediately proximal to the start codon (Hellen and Sarnow, 2001; Jang, 2006). Along the same lines, poxviruses contain a cis-acting poly A sequence in their 5′ leader that facilitates association of the pre-initiation complex with viral mRNA (Shirokikh and Spirin, 2008).

While a variety of expressions systems are known, the need continues to exist for constructs and processes which will enhance the production of useful proteins and polypeptides.

### SUMMARY OF THE INVENTION

The present invention relates to the discovery that a nucleocapsid protein (N) from hantavirus unexpectedly increases the efficiency of eukaryotic mRNA translation nonspecifically. High efficiency protein expression is important for applications ranging from commercial in vitro translation kits used extensively by research labs, to industrial high yield protein preparation. By incorporating N into in vitro translation kits, or into eukaryotic cells used for protein production, it is possible to increase protein yield.

Accordingly, in one embodiment, the invention provides a nucleotide construct comprising:

(1) a 5′ untranslated region (UTR) of RNA from a virus of the family Bunyaviridae which serves as a translational promoter for high level translation of a gene product and which binds to a Bunyaviridae nucleocapsid or active polypeptide portion thereof;
(2) a nucleotide region which begins at the 5′ end with the nucleotides AUG and which expresses a gene product protein or polypeptide; and
(3) a start codon comprising a nucleotide sequence (preferably about five or six nucleotides in length) which permits translation of the gene product starting at the AUG site.

Embodiments of the invention increase the production of a wide range of polypeptides or proteins in a production cell. Some embodiments comprise delivering (through transient transfection, stable transfection, viral vector systems, or other means) to the cell a nucleotide construct with a 5′ untranslated region (UTR). The UTR serves as a translational promoter, for high level translation of a gene product, a nucleotide region beginning (at the 5′ end) with the nucleotides AUG, which expresses a gene product protein or polypeptide, and a start codon comprising a nucleotide sequence, preferably, about 5-6 nucleotides, which permits translation of the gene product starting at the AUG site. Optionally, nucleotide constructs of the invention also comprise a spacer nucleotide group between the translational promoter and the start codon of about 0 to 40 nucleotide units. A trans-acting translational activator, a hantavirus nucleocapsid protein (N), for example Sin Nombre virus or Andes virus or an active polypeptide portion thereof (which represents an active subset of the complete nucleocapsid protein), or N or an active polypeptide portion from other viruses of the Bunyaviridae family, serves to enhance the translation of the gene product. N may be provided to the cell in the same nucleotide construct described above, in a separate nucleotide construct comprising a gene that is operably linked to a promoter such that N is expressed in the cell, or N may be delivered to the production cell as a protein or polypeptide directly. N functions in the cell in conjunction with the 5′UTR as described herein to enhance translation of the gene product resulting in an unexpectedly large increase in protein or polypeptide production from the production cell.

In another embodiment, the invention provides processes that significantly increase (e.g. in some embodiments by a factor of around fifteen) the expression of a variety of cellular and viral proteins and peptides (including but not limited to bioactive agents and food products) by using a nucleocapsid protein of hantaviruses as an initiation factor in the translation of mRNA. Certain embodiments of these processes use eukaryotic cells which have been transformed or transfected (e.g. through transient transfection, stable transfection, viral vector systems, or other means) by nucleotide constructs as described above.

Other embodiments of the processes of the invention use eukaryotic cells which have been transformed or transfected by N and a nucleotide construct which lacks a hantavirus RNA UTR translation promoter and which comprises a sequence which expresses a gene product (polypeptide or protein) in the presence of the translational enhancer N. This alternative approach also enables enhanced production of gene product from the production cell(s), although not at as high a level as the approach where both the UTR translation promoter and N are used to enhance production.

In certain embodiments, the present invention is directed to a method for increasing the production of a gene product from a production cell comprising delivering to said production cell a nucleotide construct with a nucleotide sequence, which encodes a promoter operably linked to a nucleotide sequence from members of the Bunyaviridae family.

Other embodiments includes translational enhancers in the form of other Bunyaviridae N peptides or active polypeptide portion thereof, and a nucleotide region which expresses said gene product.

Methods of producing proteins and polypeptides using the present invention result in increased production of a huge number of different gene products, including numerous polynucleotide products, and a vast number of proteins or polypeptides, including bioactive agents and food products.

Eukaryotic cells which have been transformed or transfected by a nucleocapsid protein of hantaviruses, and in vitro translation kits comprising a nucleocapsid protein of hantaviruses, are also provided.

As described herein, expression of hantavirus nucleocapsid protein (N) (the translational enhancer) by itself results in augmented translational expression of diverse genes. The mechanism of this augmentation relies on the ability of N to replace the cellular cap-binding complex, eIF4F, to attain more efficient translation initiation. This results in high level mRNA translation and greater protein/polypeptide production. We also found that inclusion of a 5′ untranslated leader region (UTR) from a hantavirus RNA, in combination with N leads to even more robust expression. In this way, the viral UTR serves as a translational promoter. The mechanism of action appears to involve recognition of the viral UTR (promoter) by N (enhancer) to provide even more robust protein production. Thus, a general strategy for expression of any gene would be to generate significant quantities of mRNA containing the viral UTR from a strong transcriptional promoter, and then to allow translation of the resulting mRNA in the presence of N (the translational enhancer). Even a modest increase in the production of commercially desirable proteins is a goal in industry. As hantaviruses are members of the Bunyaviridae family, it is to be expected that N and the UTR of additional or all members of this virus family will similarly function as translational enhancers and promoters.

As explained herein, we have discovered that the expression of N in cells appeared to surprisingly result in increased expression of heterologous indicator mRNAs. In the course of experiments to examine hantavirus nucleocapsid (N) protein function, we noted that the expression of N in cells appeared to surprisingly result in increased expression of heterologous indicator mRNAs. Here, we describe this phenomenon in detail. N can replace the activities of eIF4F to mediate mRNA translation. In particular, N binds with high affinity to the capped 5′ end of viral mRNAs, an activity that mimics that of eIF4E. N substitutes for the standard requirement for the bridging peptide, eIF4G, by directly recruiting the 43S pre-initiation complex to the 5′ mRNA cap. Finally, N replaces the helicase, eIF4A, in the cap-binding complex. Thus, this viral strategy is the functional complement to that of an IRES. N supplants the eIF4F complex in trans, whereas an IRES replaces cap-dependent translation in cis.

These and/or other aspects of the invention are described further in the following detailed description.

### BRIEF DESCRIPTION OF THE FIGURES

FIG. 1 illustrates that N protects capped 5′ termini. Aspects of FIG. 1 are explained in more detail hereinafter, e.g., in Example 1.

FIG. 2 illustrates that N associates with P bodies. Aspects of FIG. 2 are explained in more detail hereinafter, e.g., in Example 2.

FIG. 3 illustrates that N sequesters 5′ caps in P bodies. Aspects of FIG. 3 are explained in more detail hereinafter, e.g., in Examples 3 and 5.

FIG. 4 illustrates the use of mRNA and nsRNA caps in viral mRNA initiation. Aspects of FIG. 4 are explained in more detail, e.g., in Example 5.

FIG. 5 illustrates cap-snatching and translation initiation by N. Aspects of FIG. 5 are explained in more detail hereinafter, e.g., in Example 6.

FIG. 6 illustrates that N increases the expression of reporter proteins. Aspects of FIG. 6 are explained in more detail hereinafter, e.g., in Example 7.

FIG. 7 illustrates that N augments the translational expression of capped mRNA. Aspects of FIG. 7 are explained in more detail hereinafter, e.g., in Examples 7 and 8.

FIG. 8 illustrates that N binds to capped oligoribonucleotides. Aspects of FIG. 8 are explained in more detail hereinafter, e.g., in Example 8.

FIG. 9 illustrates that N preferentially augments the translation of viral mRNA. Aspects of FIG. 9 are explained in more detail hereinafter, e.g., in Example 8.

FIG. 10 illustrates that N interacts with the pre-initiation complex. Aspects of FIG. 10 are explained in more detail hereinafter, e.g., in Example 9.

FIG. 11 illustrates that N binds directly to the small ribosomal subunit. Aspects of FIG. 11 are explained in more detail hereinafter, e.g., in Example 9.

FIG. 12 illustrates that N replaces eIF4G. Aspects of FIG. 12 are explained in more detail hereinafter, e.g., in Example 9.

FIG. 13 illustrates that N promotes ribosome loading. Aspects of FIG. 13 are explained in more detail hereinafter, e.g., in Example 9.

FIG. 14 illustrates that N functionally replaces eIF4A. Aspects of FIG. 14 are explained in more detail, e.g., in Example 10.

FIG. 15 illustrates mRNA's used in the experiments of Example 8. Aspects of FIG. 15 are explained in more detail, e.g., in Example 8.

FIG. 16 illustrates certain of the results of competitive translation experiments of Example 8. Aspects of FIG. 16 are explained in more detail, e.g., in Example 8.

FIG. 17 shows mRNAs containing deletions in the viral UTR. The top sequence shows the nucleotides derived from the viral RNA template. Viral mRNAs contain heterologous 5′ caps derived from the process of cap-snatching. Thus, the remainder of the mRNAs contain an arbitrary nine nucleotide nonviral sequence appended to the 5′ end of the templated sequence to mimic the presence of a nonviral 5′ cap acquired from cellular mRNAs by cap-snatching. mRNAs devoid of various nucleotides in the viral UTR are depicted below the complete sequence.

FIGS. 18-19. Shows the competition translation analysis of some of the deletion derivatives shown in FIG. 17. In each case, the various mRNAs lacking portions of the UTR were added to translation reactions along with competitor mRNA containing a nonviral UTR and the n gene. Numbers denote the specific mRNAs displayed in FIG. 17.

FIG. 20. Illustrates binding of capped and uncapped tetrameric RNA with N. Aspects of FIG. 20 are explained in more detail hereinafter, e.g., in Example 3.

FIG. 21 Illustrates that the interaction of N with free cap is at least three orders of magnitude weaker than that observed for N with capped penta- or hexanucleotide RNA. Aspects of FIG. 21 are explained in more detail hereinafter, e.g., in Example 8.

FIG. 22 illustrates that there was no detectible stable association between N and PABP. Aspects of FIG. 22 are explained in more detail hereinafter, e.g., in Example 9.

### DETAILED DESCRIPTION OF THE INVENTION

Unless otherwise specified, “a,” “an,” “the,” and “at least one” are used interchangeably and mean one or more than one.

The following definitions may be used to describe the present invention:

The term “effective” is used to describe an amount of a component of the present invention which is used for producing an intended effect in the present invention.

The term “gene expression” is used to describe the production of a biological product encoded by a nucleic acid sequence, such as a gene sequence as otherwise described herein. This biological product, referred to herein as a “gene product,” may be a polynucleotide, but is generally a protein or a polypeptide. The polypeptide gene product is a peptide or protein that is encoded by the coding region of the gene, and is produced during the process of translation of the mRNA as otherwise described herein. Gene products which are produced according to the present invention include any protein or polypeptide which may be produced using genetic engineering methods, including bioactive, drugs and food products or additives.

The term “expression vector”, “expression construct” or “nucleotide construct” is a plasmid or other nucleotide construct such as naked DNA or naked RNA, or a viral vector, that is used to introduce and express a specific gene into a target cell. Expression vectors allow production of translatable mRNA. Once the expression vector is inside the cell, the protein that is encoded by the gene is produced by the cellular transcription and translation machinery. In the present invention, the plasmid is engineered such that it contains a highly active transcriptional promoter which causes the production of large amounts of mRNA. Preferred vectors for use in the present invention include viral vector systems such as retroviral-based or adenovirus-based vectors systems or any vector system that can produce the desired protein with a preceeding translational promoter, and in the presence of the translational enhancer (N), which is expressed either from the same vector as the desired protein or from a second vector.

The term “expression cells” is used to describe a yeast, animal or plant cell which is engineered using the nucleotide constructs which are disclosed herein to produce a protein or polypeptide gene product.

As used herein the term “operably linked” to a transcriptional promoter refers to the cloning of a gene sequence downstream to the promoter, which allows the promoter to direct the transcription of the target sequence. A linker sequence may be optionally inserted in between the promoter and the target sequence, so long as the linker sequence does not markedly hinder the performance of the reporter construct in reporting the presence of a virus.

As used herein the term “cell” refers to a eukaryotic or a prokaryotic cell, wherein the cell is capable of being transfected with one or more recombinant expression constructs so as to produce a gene product, i.e., a protein or polypeptide as otherwise described herein. In addition, production of a gene product can occur in vitro, in cellular extracts, such as rabbit reticulocyte extracts, among numerous others.

Recombinant DNA molecules, such as the recombinant expression construct used in the practice of the present invention and the like, may be isolated and synthesized using standard cloning methods such as those described by Sambrook et al. (Molecular Cloning: A Laboratory Manual, Third Edition, Cold Spring Harbor, N.Y., 2001; Current Protocols in Molecular Biology from John Wiley & Sons Inc.).

Hantaviruses are emerging pathogens, contain a tripartite negative-sense RNA genome, and comprise a genus of the Bunyaviridae family. The viral nucleocapsid protein (N) functions in both genome replication and RNA encapsidation. All viruses use the cellular translational machinery for expression of their mRNAs, and the eIF4F cap-binding complex mediates the initiation of cellular mRNA translation. The eIF4F complex is composed of eIF4E, which binds to the mRNA cap, eIF4G, which indirectly links the mRNA cap with the 43S pre-initiation complex, and eIF4A, which is a helicase required for eIF4F function. Surprisingly, we find that N has multiple intrinsic activities that mimic and substitute for each of these three peptides of the cap-binding complex thereby enhancing the translation of viral mRNA. N binds with high affinity to the mRNA cap, and to the 43S pre-initiation complex facilitating loading of ribosomes onto capped mRNA, functionally replacing eIF4E and eIF4G, respectively. Moreover, N substitutes for the helicase, eIF4A. The expression of a multifaceted viral protein that functionally supplants the cellular translation initiation complex is a unique strategy for viral mRNA translation initiation. The ability of N to directly mediate translation initiation would be expected to circumvent some mechanisms of intracellular immunity, and also allow the virus to avoid the negative regulatory effect of proteins that interact with the cellular cap-binding complex, ensuring the efficient translation of viral mRNA. Thus, this viral strategy is the functional complement to an IRES. N supplants the eIF4F complex in trans, while an IRES replaces cap-dependent translation in cis.

A defined-sequence protein replaces the entire cellular eIF4F complex. The eIF4F cap-binding complex mediates the initiation of cellular mRNA translation. eIF4F is composed of eIF4E, which binds to the mRNA cap, eIF4G, which indirectly links the mRNA cap with the 43S pre-initiation complex, and eIF4A, which is a helicase necessary for initiation. Viral nucleocapsid proteins (N) function in both genome replication and RNA encapsidation. Surprisingly, we find that hantavirus N has multiple intrinsic activities that mimic and substitute for each of the three peptides of the cap-binding complex thereby enhancing the translation of viral mRNA. N binds with high affinity to the mRNA cap replacing eIF4E. N binds directly to the 43S pre-initiation complex facilitating loading of ribosomes onto capped mRNA functionally replacing eIF4G. Finally, N obviates the requirement for the helicase, eIF4A. The expression of a multifaceted viral protein that functionally supplants the cellular cap-binding complex is a unique strategy for viral mRNA translation initiation. The ability of N to directly mediate translation initiation would ensure the efficient translation of viral mRNA.

### A. Translational Promoter.

Preferred nucleotide constructs according to the present invention comprise a translational promoter according to the formula:

m7G(X)₀₋₁₅(UAG)₂₋₅(X)₀₋₄₀(start codon nucleotides)₅₋₆AUG−polynucleotide encoding gene product(remainder of the gene)

where m7G is the cap of the promoter;
X is a nucleotide (preferably a nucleotide—including 2′-deoxynucleotides—containing a base selected from the group consisting of guanine, adenine, cytosine, uracil and inosine, or other potential ribonucleotides that can be incorporated into RNA);
UAG is one copy of the triplet repeat
start codon nucleotides=any combination of nucleotides that permit translation of the gene at the 5′AUG nucleotide triplet of the gene; and AUG=beginning of the gene to be translated into protein.

The cis-acting translational promoter as set forth above is recognized by the Bunyaviridae (preferably hantavirus) nucleocapsid N, which is described in greater detail herein.

The 5′ UTR of the Bunyaviridae (hantavirus) mRNA serves as a “translational promoter” for high level translation and protein production when trans-activated by the nucleocapsid protein, the translational enhancer. The following are preferred aspects of this translational promoter activity:
1. The 5′ end of the mRNA has a m7G cap (7-methyl guanosine). Cap analogs such as 2-O-methyl G7 do not work well for promoter activity.
2. The promoter must contain two or more of the terminal triplet repeats present in viral mRNA, or any sequence that serves as a high affinity binding site for hantavirus nucleocapsid protein (N). The minimal triplet repeat sequence is UAGUAG.
3. Following the triplet repeat, and immediately proximal to the start codon (AUG) of the gene to be expressed, the promoter must contain five or six nucleotides sufficient for efficient start codon usage (start codon context nucleotides). The sequence of these five or six nucleotides can vary but will typically be composed of a minimal kozak-type of sequence, or a sequence that permits normal expression from the start codon.
4. A variable number of additional nucleotides (up to 40) can also be present between the triplet repeat sequences and the kozak-type sequences.
5. A variable number of nucleotides of variable sequence can be placed between the m7G cap and the triplet repeat (e.g. up to 15).

Some variation of the translational promoter will allow N-mediated trans-activation of protein production. These include the following:

1. Variants of the triplet repeat that can mutate but still be recognized by N at high affinity.
2. The presence of the triplet repeat near an internal ribosomal entry sequence (IRES) allows N to be recruited to the IRES.

Other UTR sequences from the mRNAs from all other members of the Bunyavirus family including the hantaviruses, orthobunyavirus, nairovirus, tospovirus, and phlebovirus genera, that can be recognized with high affinity by N from the corresponding virus.

B. The Trans-Acting Translational Activator (i.e. Translational Enhancer) Nucleocapsid Protein N.

The Bunyaviridae (e.g., hantavirus) nucleocapsid protein (N) serves as a high level translational activator (enhancer) of translation. This activity of N works independently of the promoter described in A. However, use in conjunction with the translational promoter (described in A) enables optimal translation and protein production. The following are the requirements for the activity of N:

1. An mRNA with the characteristics outlined above in “A. Translational Promoter”, or an mRNA with a cap.
2. A preferred sequence of N is SEQ ID NO: 1:

MSTLKEVQDNITLHEQQLVTARQKLKDAERAVELDPDDVNKSTLQSRRA
AVSALETKLGELKRELADLIAAQKLASKPVDPTGIEPDDHLKEKSSLRY
GNVLDVNSIDLEEPSGQTADWKSIGLYILSFALPIILKALYMLSTRGRQ
TIKENKGTRIRFKDDSSYEEVNGIRKPRHLYVSMPTAQSTMKADEITPG
RFRTIACGLFPAQVKARNIISPVMGVIGFSFFVKDWMERIDDFLAARCP
FLPEQKDPRDAALATNRAYFITRQLQVDESKVSDIEDLIADARAESATI
FADIATPHSVWVFACAPDRCPPTALYVAGMPELGAFFAILQDMRNTIMA
SKSVGTSEEKLKICKSAFYQSYLRRTQSMGIQLDQKIIILYMSHWGREA
VNHFHLGDDMDPELRELAQTLVDIKVREISNQEPLKL*

In one embodiment, an amino acid sequence (designated as SEQ ID NO:2) comprising the first 175 amino acids of SEQ ID NO. 1 (designated as SEQ ID NO:2) proves sufficient for the translation activation activity of N, provided residues are held in trimeric form by either the appropriate additional regions of N, or by a foreign trimerization peptide:

MSTLKEVQDNITLHEQQLVTARQKLKDAERAVELDPDDVNKSTLQSRRAAVSALET KLGELKRELADLIAAQKLASKPVDPTGIEPDDHLKEKSSLRYGNVLDVNSIDLEEPSG QTADWKSIGLYILSFALPIILKALYMLSTRGRQTIKENKGTRIRFKDDSSYEEVNGIRK P (SEQ ID No: 2) which is linked to a trimerization domain or SEQ ID NO: 2—trimerization domain.

In other embodiments, translational activity of N is achieved by a sequence that is homologous to any of SEQ ID Nos. 1 and 2, preferably a polypeptide that has an amino acid sequence at least 60%, or in some embodiments at least 70%, or in some embodiments at least 80%, or in some embodiments at least 80%, or in some embodiments at least 95% homologous to the polypeptides represented by SEQ ID Nos: 1 or 2.

It is to be expected that some variation of the translational activator (N) will allow trans-activation of protein production. Therefore, trans-activation of protein production should also be achievable through use of:

1. a subset of the amino acids of N sufficient for activity (in trimeric form); and
2. the N peptide from other members of the Bunyavirus family including the hantaviruses, orthobunyavirus, nairovirus, tospovirus, and phlebovirus genera.

Useful peptide sequences for trans-activation of protein production also include, but are not limited, to, inter alia, the amino acid sequences which define the proteins listed below, as well as a polypeptide that has an amino acid sequence that is at least 60%, or in some embodiments at least 70%, or in some embodiments at least 80%, or in some embodiments at least 80%, or in some embodiments at least 95%, 96%, 97%, 98%, 99% or 99.5+% homologous to the amino acid sequences of the proteins listed below:

nucleocapsid protein [Rio Mamore hantavirus] AAC58449;
nucleocapsid protein [Rio Mamore hantavirus] AAC58448;
nucleocapsid protein [Hantavirus HTN-007] AAD54772;
nucleocapsid protein [El Moro Canyon hantavirus] AAD09463;
nucleocapsid protein [El Moro Canyon hantavirus] AAD09462;
nucleocapsid protein [El Moro Canyon hantavirus] AAD09461;
nucleocapsid protein [Hantavirus HTN/Far East/4226] AAF02672;
nucleoprotein [Central Plata virus] ACF05496;
Maguari virus N AAA57147;
N protein [Shokwe virus] ACE07184;
N protein [Xingu virus] ACE07182;
N protein [Pongola virus—SAAr1] ACE07178;
Nucleocapsid protein Dugbe virus [P15190];
nucleocapsid protein [Crimean-Congo hemorrhagic fever virus] AAQ23152;
nucleoprotein N [Physalis severe mottle virus] AAD34201;
Nucleocapsid protein [P22025];
nucleocapsid [Phlebovirus sp. PAN 483391 [ ABQ23576]; and
nucleoprotein [Toscana virus] [ACM92017].

### C. Production Cells.

The combination of translational promoter (described in A) and translational trans-activator (described in B) will work in diverse mammalian cells, so it is to be expected that all mammalian cells can potentially serve as production cells for generation of the protein or proteins of interest.

Production can also be achieved in vitro in rabbit reticulocyte extracts

### D. Delivery.

Both the translational promoter (and its associated gene), and the translational trans activator (enhancer) (hantavirus N gene), can be delivered to cells via DNA transfection protocols. Additionally, both components:

1. can be delivered to cells by way of any DNA or RNA transfection systems;
2. can be delivered to cells by way of any viral vector system such as retroviral-based or adenovirus-based vector systems, or any vector system that can express N and a gene under the control of the translational promoter; and
3. can be delivered to cells together on the DNA or RNA during transfection, or on the same vector. Alternatively, the components can be delivered to cells on separate DNAs, or RNAs, or vectors.

Additionally, the following are aspects of the invention.

1. Hantavirus N, by itself, significantly boosts the translational expression of any gene of interest from capped mRNAs.
2. Hantavirus N, when used along with a gene containing the viral 5′ UTR allows more robust gene expression than the expression achieved using only N.
3. Hantaviruses are members of the Bunyavirus family of viruses. It is to be expected that the N peptides and 5′ UTRs of some or all members of the family will have the same effect.
4. Hantaviruses are negative sense segmented RNA viruses. Given the similarity between the general replication schemes of all such viruses, including the orthomyxovirus, arenaviruses, and bunyaviruses, it is to be expected that the nucleocapsid peptides (N or NP) will have similar activity.
5. Given the similar roles of other N peptides from RNA viruses, including those of the paramyxo- and rhabdoviruses, it is to be expected that the N peptides from these virus families will have similar activities.
6. Not only does Hantavirus N significantly boosts the translational expression of any gene of interest from capped mRNAs, hantavirus N peptide will increase the expression from internal ribosomal entry sequences (IRESes).
7. N, with or without the viral UTR, will increase the expression of any gene in eukaryotic cells. However, expression is optimal when both N and the viral UTR are present.
8. N, with or without the viral UTR, will increase the expression of any gene in rabbit reticulocyte or other extracts used for in vitro translation.
9. The combination of N and the viral UTR, or N alone, will work in situations where appropriate RNA is supplied directly.
10. The combination of N and the viral UTR, or N alone, will work in situations where appropriate RNA is expressed from a transcriptional promoter provided endogenously in cells, or through transfection.

The invention is described further in the following experimental section and examples, which are illustrative only and in no way limiting.

### EXPERIMENTAL SECTION

A further description of the materials and methods used in Experiments 1-10 is found after the “Discussion of Experimental Results”.

### Example 1

### Binding of Hantavirus N to mRNA Caps

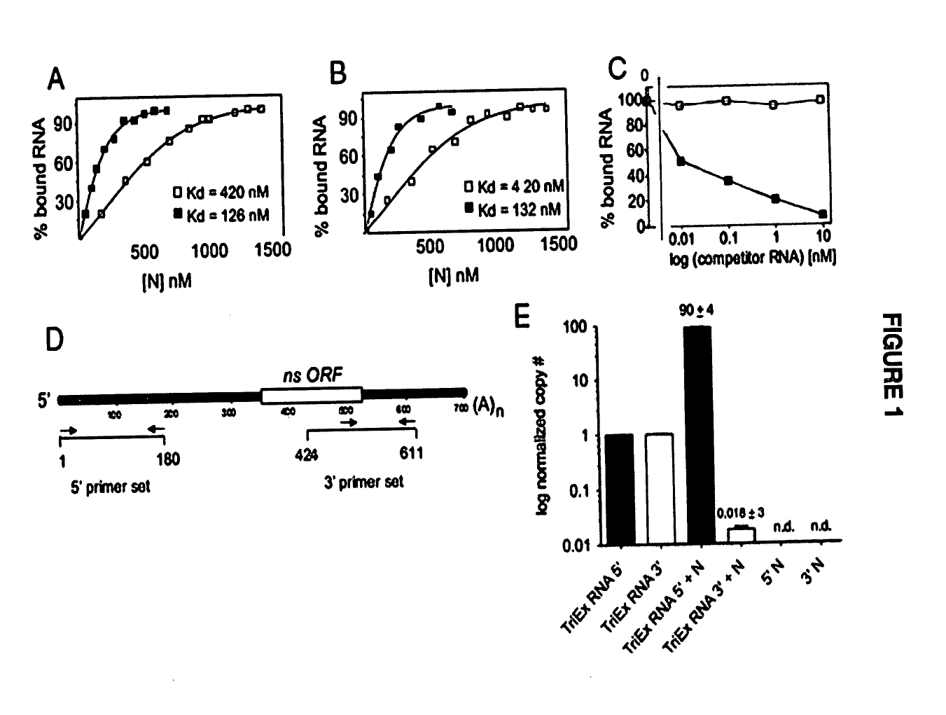

During the course of experiments to examine RNA recognition by hantavirus nucleocapsid protein (N) we observed that N preferentially binds to RNA containing 5′ caps compared with uncapped RNA. To further examine this association we synthesized a labeled RNA ′600 nucleotides in length from pTriEX, containing a random ORF, that either contained or lacked a 5′ cap, and carried out RNA filter binding experiments with increasing amounts of N. N interacted with capped RNA with three to four fold higher affinity than with uncapped RNA (FIG. 1A). Moreover, N interacted at similar affinity with a short RNA corresponding to the 5′ terminal 10 nucleotides of this RNA provided this oligonucleotide was capped, indicating that a short capped RNA can be recognized by N (FIG. 1B). To further explore the interaction of N with capped RNA we carried out competition analysis of a capped labeled decamer RNA (m7GTCTCTCCCA) with increasing concentrations of unlabeled decamer competitor RNA capped with m7G or the cap analogue 2′-O-methyl G. This indicated that the m7G, but not the 2′-Omethyl G, oligomeric RNA inhibited binding of N to the labeled capped decamer (FIG. 1C).

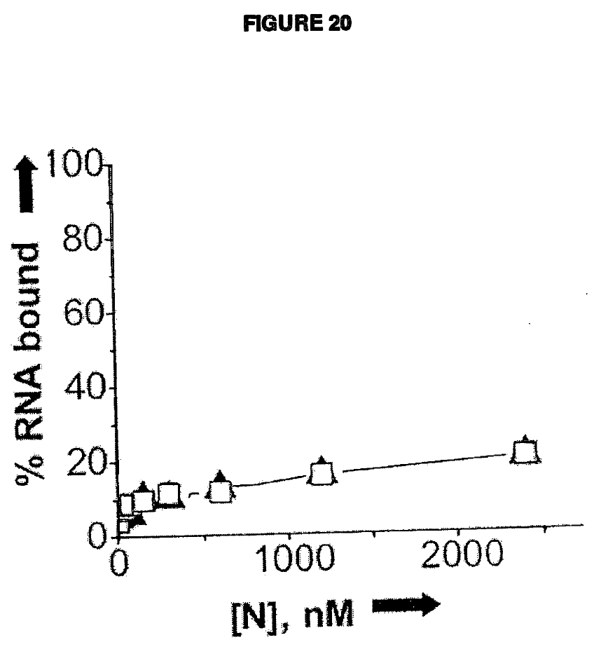

Because N bound short RNAs with 5′ caps in vitro, we wanted to determine whether N protects the 5′ caps of mRNAs in cells. Thus, we compared the in vivo stability of an mRNA in the presence and absence of N. pTriEx, which can also express an RNA 703 nucleotides in length from a eukaryotic promoter-enhancer (FIG. 1D), was transfected into HeLa cells along with a plasmid that expresses N. Thirty-six hours after transfection we isolated RNA from the transfected cells and used quantitative real-time PCR to measure the relative intracellular abundance of the 5′ and 3′ regions of this RNA. This analysis indicated that the 5′ end of the RNA was markedly more abundant in the presence of N, and also that the 3′ end was strikingly scarce in the presence of N (FIG. 1E). These data are consistent with the idea that N protects the 5′ end of capped mRNA from degradation through binding to 5′ caps, and that N diminishes the steady-state level of the 3′ end through an unknown mechanism. It should be noted that all real-time PCR reactions contained an internal control to measure B-actin mRNA. N did not affect the steady-state level of this mRNA (See discussion of FIG. 20 hereinafter).

Further details of FIG. 1 and the experiment(s) which yielded the data reflected in that figure are as follows.

### FIG. 1.

(A) Binding of N to synthetic capped and uncapped TriEx RNA was examined by using radiolabeled RNA and filter binding with increasing concentrations of N as described in Materials and Methods. Dissociation constants (Kd) are indicated. Solid squares indicate capped RNA, open squares indicate uncapped RNA. (B) Parallel binding of N to a capped or uncapped synthetic decamer RNA corresponding to the 5′ terminus of TriEx RNA was examined using filter binding as in A. Solid squares indicate capped RNA, open squares indicate uncapped RNA. (C) Competition binding analysis using m7GTCTCTCCCA labeled with P32 CTP and unlabeled GTCTCTCCCA with either an m7 or 2′-Omethyl cap. This oligonucleotide was chosen to ensure quantitative capping as the sequence lacks internal G residues. N binds with this capped decamer RNA at an affinity (Kd′ 130 nM) similar to that of the decamer in B. Reactions contained 0.01 nM of labeled decamer, 520 nM N, and increasing amounts of competitor RNA containing an m7 or 2′-O-methyl cap as indicated in the log scale. The amount of RNA binding in the absence of competitor (100% binding) is also depicted for clarity, although [0 nM] competitor cannot be plotted on a log scale. Closed and open squares indicate cold competitor decamer with an m7 cap or 2′-O-methyl cap, respectively. (D) Diagram of TriEx RNA expressed in transfected HeLa cells. Following cDNA synthesis by reverse transcriptase, real-time PCR was used to quantify the 5′ and 3′ ends using primers complementary to the indicated nucleotides. See Examples 1-6 Materials and Methods, infra, for a detailed description of quantitative real-time PCR and exact primer sequences. The RNA encodes a short, arbitrary ORF. (E) Effect of co-expression of N on the 5′ and 3′ ends of TriEx RNA. The quantified PCR products of the 5′ and 3′ termini in the absence of N were used for normalization.

### Example 2

### Association of Hantavirus N with P Bodies

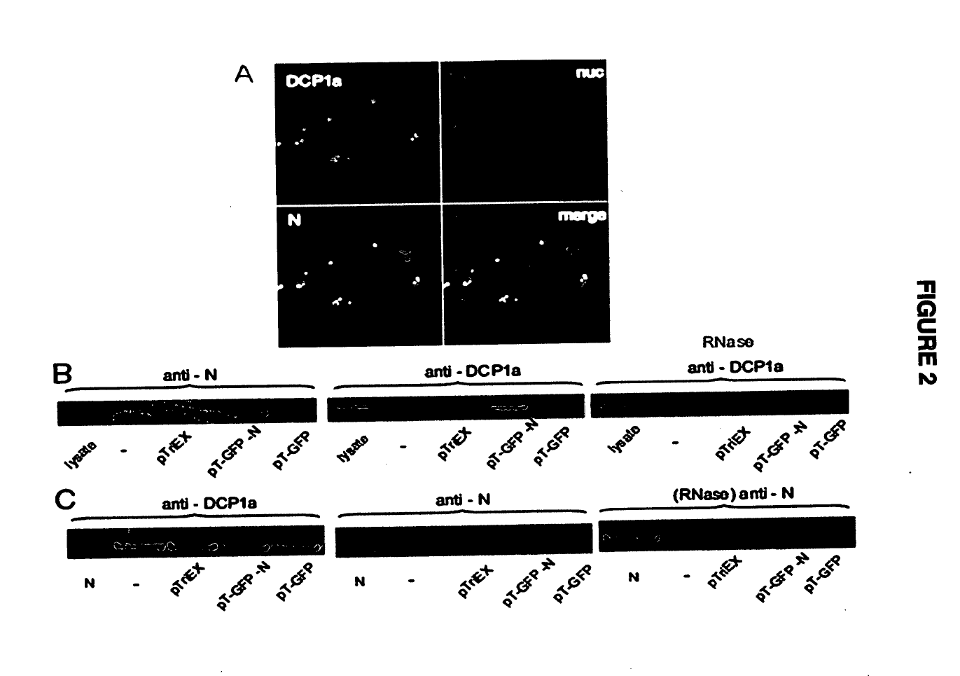

It seemed feasible that N-mediated protection of the 5′ ends of capped mRNAs could take place in P bodies, where 5′ capped RNAs might be stored for later use. Thus, we tested the idea that N-cap complexes preferentially reside in cytoplasmic P bodies. To track intracellular N, we transfected HeLa cells with a plasmid expressing an N fusion peptide flanked on its N terminus with GFP (pT-GFP-N). In addition, this N fusion peptide contained an octahistidine tag on its C terminus to facilitate its recovery from cells. Using confocal microscopy, P bodies were visualized by using a monoclonal antibody specific for DCP1, a signature peptide of P bodies (19-21). Significantly, the N fusion peptide strongly co-localized with DCP1 in P bodies (FIG. 2A).

In a complementary assay, we transfected cells with pTGFP-N, pTriEx (a empty vector control), or pT-GFP (a control that expresses GFP with a C-terminal octahistidine tag). We then isolated N from HeLa cell lysates by using Ni-nitrilotriacetic acid (NTA) beads, which bind with the C-terminal octahistidine tag on N, and carried out Western analysis with anti-DCP1 antibody to determine whether P body components stably associate with N. The results of this experiment indicated that DCP1 interacts with N (FIG. 2B). The integrity of P bodies depends on the presence of associated RNA, as loss of the RNA from P bodies results in dissociation of the proteins resident in P bodies (22). Notably, RNase A digestion of cell lysates before recovery of N on Ni-NTA beads resulted in marked reduction in co-association of DCP1 (FIG. 2B). We used a complementary co-immunoprecipitation assay to further verify association of N with P bodies. P bodies were immunoprecipitated from HeLa cell lysates using a monoclonal antibody against DCP1 and recovery with Sepharose G beads. The immunoprecipitated samples were then monitored for co-precipitation of N using a Western blot with polyclonal anti-N antibody. This experiment again indicated that N is associated with P bodies (FIG. 2C), and RNase A treatment of the lysate before immunoprecipitation verified that association between N and DCP1 is RNA-dependent (FIG. 2C).

Further details of FIG. 2 and the experiment(s) which yielded the data reflected in that figure are as follows.

### FIG. 2.

(A) Confocal detection of cytoplasmic P bodies and N. HeLa cells were transfected with plasmid expressing a GFP-N fusion protein. Intracytoplasmic DCP1 was detected with anti-DCP1 antibody. N was visualized by detection of GFP, and nuclei by DAPI. (B) Pull-down analysis to detect association of N with P bodies. N was recovered from the lysates of transfected cells by virtue of a C-terminal octahistidine tag using Ni-NTA columns. Recovered material was analyzed with Western blots with anti-N antibody to verify recovery of N and with anti-DCP1 to detect association of P body components with N. The indicated samples were treated with RNase A before recovery to verify that association of DCP1 with N was RNA-dependent. Dashes represent untransfected cells. Lysate (sample from pTriEX transfected cells before fractionation), pTriEx (an empty vector control), pT-GFP-N (a plasmid that expresses a GFP-N fusion peptide), and pT-GFP (a negative control plasmid that expresses the GFP portion of pT-GFP-N but that lacks N) are described in the text. (C) Communoprecipitation analysis to further verify association of N with P bodies. DCP1 was recovered by immunoprecipitation with anti-DCP1 Ab and Sepharose-G beads. Recovered material was examined by Western analysis with anti-DCP1 Ab to verify recovery of DCP1, or with anti-N Ab to detect co-precipitation of N with DCP1. As in B, some samples were also treated with RNase A before recovery to verify that association between N and DCP1 is RNA-dependent. (n represents purified bacterially expressed N; dashes represent untransfected cells; pTriEx, pT-GFP-N, and pT-GFP are as described for B.)

### Example 3

### Preferential Protection of Caps from an mRNA Containing a PTC

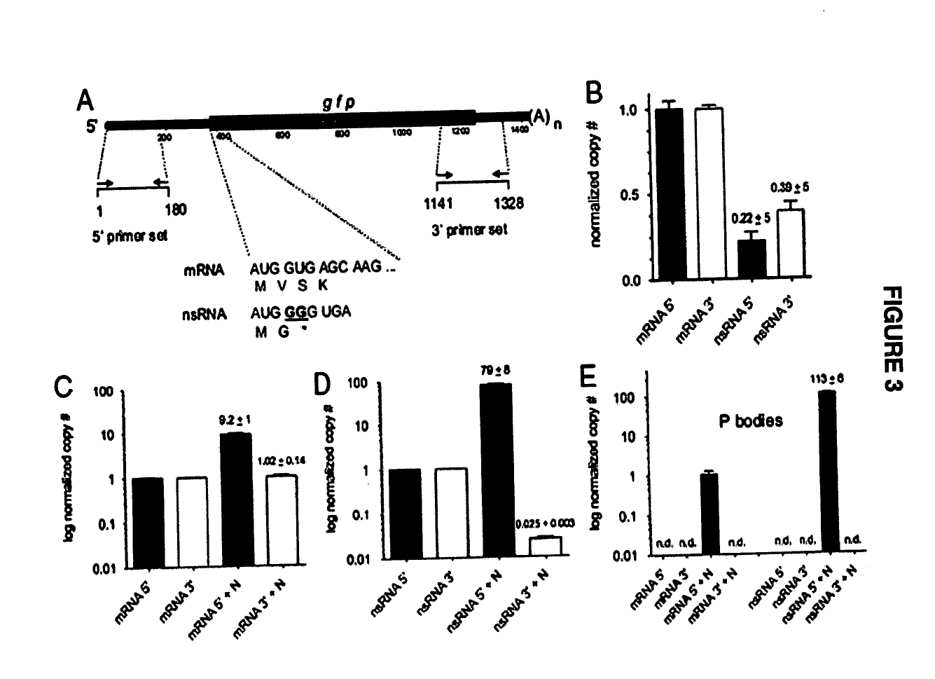

The RNA used in FIG. 1 to examine N-mediated stabilization of 5′ caps expresses a peptide from an arbitrary ORF. Such an RNA might be targeted for degradation by the NMD. Thus, we wanted to compare the intracellular abundance and distribution of a functional mRNA with that of a related “nonsense RNA” (nsRNA) containing a PTC, and determine the effect of N on the stability of both RNAs. pT-GFP expresses a functional mRNA that is translated into GFP. pT-GFPns expresses an nsRNA containing a two-nucleotide insertion at nucleotide position 4 of the GFP gene. nsRNA would be translated into a dipeptide and terminate at a stop codon arising from the frame shift (FIG. 3A).

As expected, the steady-state levels of both the 5′ and 3′ termini of this nsRNA were reduced relative to the corresponding mRNA, presumably as a result of NMD of the nsRNA (FIG. 3A). Interestingly, the presence of N increased the relative abundance of the 5′ end of both the mRNA and the nsRNA. However, protection of the 5′ terminus and degradation of the 3′ terminus by N was strikingly more robust for the nsRNA than the corresponding mRNA (compare FIG. 3C vs. FIG. 3D).

These data are consistent with the hypothesis that protection of the 5′ terminus of nsRNA by N is more efficient because of preferential targeting of the nsRNA to P bodies by the NMD pathway.

To directly examine and quantify 5′ caps in P bodies in the presence and absence of N, we transfected HeLa cells with either pT-GFP or pT-GFPns along with a plasmid expressing N (or an empty vector control). P body components were recovered by immunoprecipitation with monoclonal antibody against DCP1, RNA. This analysis indicated that the 5′ end of the RNA was markedly more abundant in the presence of N, and also that the 3′ end was strikingly scarce in the presence of N (FIG. 1E). These data are consistent with the idea that N protects the 5′ end of capped mRNA from degradation through binding to 5′ caps, and that N diminishes the steady-state level of the 3′ end through an unknown mechanism. It should be noted that all real-time PCR reactions contained an internal control to measure B-actin mRNA. N did not affect the steady-state level of this mRNA (FIG. 20).

### Example 4

### Association of Hantavirus N with P Bodies

It seemed feasible that N-mediated protection of the 5′ ends of capped mRNAs could take place in P bodies, where 5′ capped RNAs might be stored for later use. Thus, we tested the idea that N-cap complexes preferentially reside in cytoplasmic P bodies. To track intracellular N, we transfected HeLa cells with a plasmid expressing an N fusion peptide flanked on its N terminus with GFP (pT-GFP-N). In addition, this N fusion peptide contained an octahistidine tag on its C terminus to facilitate its recovery from cells. Using confocal microscopy, P bodies were visualized by using a monoclonal antibody specific for DCP1, a signature peptide of P bodies (19-21). Significantly, the N fusion peptide strongly co-localized with DCP1 in P bodies (FIG. 2A). In a complementary assay, we transfected cells with pTGFP-N, pTriEx (a empty vector control), or pT-GFP (a control that expresses GFP with a C-terminal octahistidine tag). We then isolated N from HeLa cell lysates by using Ni-nitrilotriacetic acid (NTA) beads, which bind with the C-terminal octahistidine tag on N, and carried out Western analysis with anti-DCP1 antibody to determine whether P body components stably associate with N. The results of this experiment indicated that DCP1 interacts with N (FIG. 2B). The integrity of P bodies depends on the presence of associated RNA, as loss of the RNA from P bodies results in dissociation of the proteins resident in P bodies (22). Notably, RNase A digestion of cell lysates before recovery of N on Ni-NTA beads resulted in marked reduction in co-association of DCP1 (FIG. 2B). We used a complementary co-immunoprecipitation assay to further verify association of N with P bodies. P bodies were immunoprecipitated from HeLa cell lysates using a monoclonal antibody against DCP1 and recovery with Sepharose G beads. The immunoprecipitated samples were then monitored for co-precipitation of N using a Western blot with polyclonal anti-N antibody. This experiment again indicated that N is associated with P bodies (FIG. 2C), and RNase A treatment of the lysate before immunoprecipitation verified that association between N and DCP1 is RNA-dependent (FIG. 2C).

### Example 5

### Preferential Protection of Caps from an mRNA Containing a PTC

The RNA used in FIG. 1 to examine N-mediated stabilization of 5′ caps expresses a peptide from an arbitrary ORF. Such an RNA might be targeted for degradation by the NMD. Thus, we wanted to compare the intracellular abundance and distribution of a functional mRNA with that of a related “nonsense RNA” (nsRNA) containing a PTC, and determine the effect of N on the stability of both RNAs. pT-GFP expresses a functional mRNA that is translated into GFP. pT-GFPns expresses an nsRNA containing a two-nucleotide insertion at nucleotide position 4 of the GFP gene. nsRNA would be translated into a dipeptide and terminate at a stop codon arising from the frame shift (FIG. 3A). As expected, the steady-state levels of both the 5′ and 3′ termini of this nsRNA were reduced relative to the corresponding mRNA, presumably as a result of NMD of the nsRNA (FIG. 3A). Interestingly, the presence of N increased the relative abundance of the 5′ end of both the mRNA and the nsRNA. However, protection of the 5′ terminus and degradation of the 3′ terminus by N was strikingly more robust for the nsRNA than the corresponding mRNA (compare FIG. 3C vs. FIG. 3D). These data are consistent with the hypothesis that protection of the 5′ terminus of nsRNA by N is more efficient because of preferential targeting of the nsRNA to P bodies by the NMD pathway.

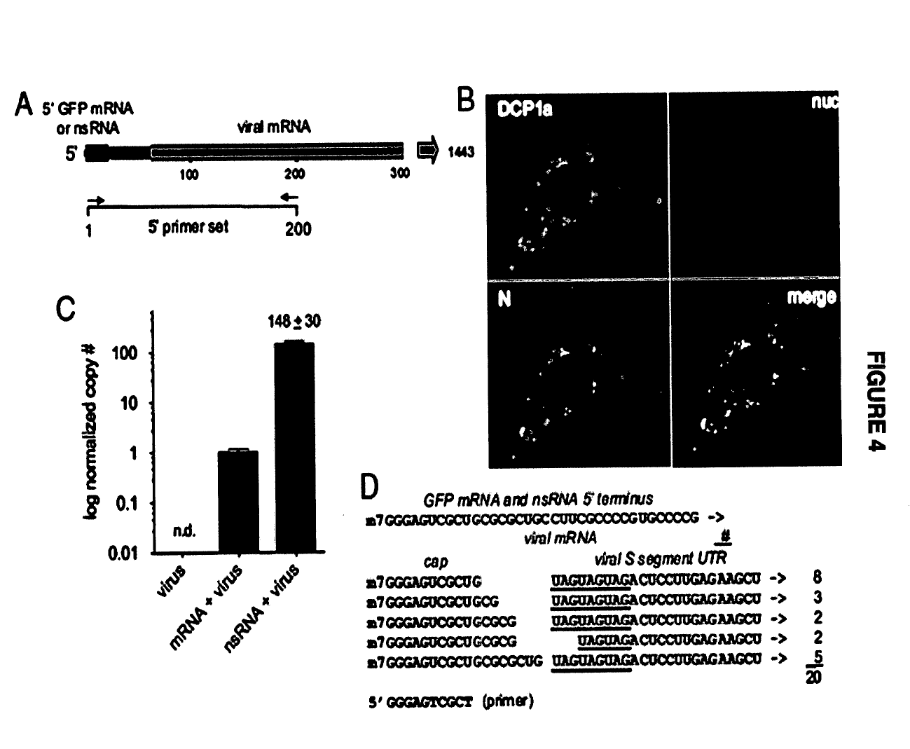

To directly examine and quantify 5′ caps in P bodies in the presence and absence of N, we transfected HeLa cells with either pT-GFP or pT-GFPns along with a plasmid expressing N (or an empty vector control). P body components were recovered by immunoprecipitation with monoclonal antibody against DCP1, several hours later, total RNA from the virus-infected cells was harvested and acquisition of caps from GFP mRNA or GFP nsRNA was quantified using a 10-bp primer corresponding to the 5′ terminus of GFP mRNA/nsRNA and a second primer complementary to SNV S segment mRNA (FIG. 4A). As expected, N from virus-infected cells strongly co-localized with P bodies as evidenced by confocal analysis using anti-DCP1 and anti-N antibody (FIG. 4B). Significantly, caps from the nsRNA were substantially more prevalent in viral mRNA than were caps from the mRNA, results that paralleled their relative abundance in P bodies (FIG. 4C).

Cap snatching by hantaviruses typically generates caps eight to seventeen nucleotides in length that preferentially terminate in a G immediately preceding two or three copies of the terminal triplet repeat in the viral UTR sequence (8). To determine whether the caps derived from GFP RNA exhibit these hallmarks of correct cap snatching, we sequenced the cap-viral UTR junctions arising from viral mRNA containing caps from nsGFP. Because the cap-specific primer used in amplification was 10 nucleotides in length, only caps greater than 10 nucleotides would be detected. Analysis of 20 randomly selected clones indicated that all had caps derived from nsGFP, ranging in length from 11 to 18 nucleotides terminating at available G residues at positions 11, 13, 15, and 18 (FIG. 4D). Taken together, all these data indicate that N plays a role in cap snatching by sequestering capped RNAs in P bodies for use by the viral RdRp during transcription initiation.

The 5′ caps of RNA containing a premature termination codon were preferentially targeted to P bodies, protected by viral N protein, and used in the initiation of viral transcription. However, we expect that the virus uses any capped RNAs that are trafficked to P bodies. This would include mRNA arising in P bodies through routine turnover, defective RNA that is transported to P bodies by the NMD pathway or similar pathways, and mRNA that is specifically targeted for degradation by pertinent cellular regulatory signals.

In addition to preservation of 5′ caps by N the 3′ terminus of nsRNA is markedly degraded in the presence of N (FIGS. 1D and 3D). We suggest that the reason for decreased stability of the 3′ end is that N inhibits circularization of nsRNA. mRNAs are circularized through interaction between eIF4G at the 5′ cap (in the eIF4F cap binding complex) and poly(A) binding protein at the 3′ end (23, 24).

It is likely that circularization through this protein bridge stabilizes mRNA. Binding of N to the 5′ cap likely inhibits concomitant binding by eIF4G, abrogating RNA circularization, leading to more efficient degradation of the 3′ end. (The 5′ end is stabilized by association with N and storage in P bodies.) A related possibility is that N increases the rate at which nsRNA is trafficked to P bodies. This might result in robust degradation of nsRNA and concomitant protection of 5′ caps by N. It is important to note that robust 3′ end degradation in the presence of N is averted by efficient mRNA translation. The steady-state level of the 3′ ends of translated mRNA is unchanged in the presence or absence of N, indicating that the pool of mRNA being translated remains constant (FIG. 3C). The approximately nine-fold increase in intracellular 5′ termini derived from the mRNA is probably a result of 5′ caps in P bodies that accrue during normal mRNA turnover when N is present to preserve those caps. Preservation of 5′ caps by N is likely mediated through simple protection of 5′ termini from decapping by DCP2/1 and subsequent 5′ to 3′ degradation by XRN1. This mechanism of protection is obviously predicated on inability for simultaneous binding by both N and DCP2/1 with the cap. N and DCP1 are co-precipitated from cells expressing N, and N-DCP1 association is abolished by RNase treatment (FIG. 2). It is likely that these two proteins are associated with separate RNAs but are intermolecularly linked through a network of one or more additional RNA binding proteins and RNA present in P bodies. However, N does bind uncapped RNA at lower affinity, so it is possible that N associates intramolecularly with DCP1 by binding to the interior of RNA molecules. N protects a minimum of 180 5′ terminal nucleotides of capped RNA in P bodies (FIG. 3 A, E).

The 5′ caps of Bunyaviruses are typically 10 to 18 nucleotides in length (7, 8) (FIG. 4D), indicating that the caps sequestered by N are further trimmed before or during transcription initiation. In influenza virus infection, caps are also approximately this length and are generated by endonuclease cleavage carried out by the RdRp. A cap-dependent endonuclease activity is present in Bunyavirus preparations (25), and it is presumed that hantavirus RdRp ultimately generates caps of appropriate length. To date, we have not detected endonuclease activity associated with N. An alternative possibility is that one or more cellular nucleases resident in P bodies is incorporated into particles and that such enzymes mediate the final trimming of primers before transcription initiation.

Further details of FIGS. 3 and 4 and the experiment(s) which yielded the data reflected in those figures are as follows.

### FIG. 3.

(A) We used an mRNA that expresses GFP, and a closely related nsRNA containing a premature termination codon to examine the effect of N on RNA stability. The GFP gene in the nsRNA contains a premature stop codon resulting from the insertion of two G residues (shown in bold). A primer pair corresponding to the first 180 nucleotides of both RNAs was used to quantify 5′ termini using real-time PCR following reverse transcription. A second primer pair was used to quantify a region near the 3′ termini of both RNAs. (B) The relative steady state levels of the 5′ and 3′ termini of the mRNA and nsRNA in the absence of N are shown. The quantified PCR products of the 5′ and 3′ termini in the GFP mRNA were used for normalization. (C) Comparison of the steady-state levels of 5′ and 3′ termini GFP mRNA in the presence and absence of N. (D) Comparison of the steadystate levels of 5′ and 3′ termini in GFP nsRNA in the presence and absence of N. (E) Effect of N on the relative abundance of 5′ and 3′ termini from GFP mRNA and nsRNA in P bodies. P body-associated material was recovered by immunoprecipitation with anti-DCP1 Ab as in FIG. 2. RNA was then prepared and the 5′ and 3′ termini quantified. 5′ termini in the absence of N were used for normalization. (n.d.: not detected.)

### FIG. 4.

(A) Composite viral mRNAs containing caps from GFP mRNA or nsRNA were detected using a sense primer matching the 5′ end of the GFP RNA and primer complementary to SNV S segment mRNA as shown. The total length of S segment mRNA is 2,076 nucleotides, not including the cap. (B) Confocal detection of cytoplasmic P bodies and N in virus-infected cells. Twenty-four hours after infection, intracytoplasmic DCP1 was detected with anti-DCP1 antibody, N was visualized by detection with anti-N antibody, and nuclei by DAPI. (C) Quantification of virus-infected cells expressing GFP mRNA or nsRNA. “Virus” represents RNA from virus-infected cells; “mRNA virus” represents RNA from virus-infected cells expressing GFP mRNA (used for normalization of the graph); and “nsRNA ‘virus”represents RNA from virus-infected cells expressing GFP nsRNA. (D) Sequence analysis of caps from GFP nsRNA on viral mRNA. RT-PCR products were cloned and 20 DNAs were randomly obtained and sequenced. Cap sequences are depicted in blue and viral UTR sequences in green. The triplet repeats present at the terminus of the viral UTR are underlined. The number of clones with each displayed sequence is indicated.

### Example 6

### Examination of the Primary Sequence of N does not Reveal Obvious Similarity or Motifs with Other Cap Binding Peptides

The three-dimensional structure of CBP20 in the nuclear cap-binding complex, of eIF4E of the eIF4F translation initiation complex, and vaccinia virus cap-binding peptide, VP39, suggests that these peptides have undergone convergent evolution to enable similar interactions with the cap (26). Specifically, each of these peptides feature two aromatic residues that form stacking interactions with the guanine cap, with an ancillary role for an acidic residue for stabilization of the interaction. Of the various combinations of aromatic residues in N, the most similar to that of eIF4E, CBP20, and VP39 are W119 and Y165E166; the spacing is identical to that in eIF4E, and Y165 features an adjacent E as in eIF4E. Identification of domains of N involved in cap protection should be useful in determining whether N is similar to other cap binding peptides.

Examination of hantavirus assembly indicates that N associates with the ER-Golgi intermediate compartment in transit to the site(s) of virus budding (27). The extensive confocal analysis used in these studies depicts N in punctate intracellular distribution. Based on the co-association between N and DCP1 we observed, these granular structures are likely to be P bodies. Virus assembly would therefore apparently involve interaction of N with both P bodies and intracellular membranes. It is not clear whether such association would occur simultaneously or whether membrane association follows P body association.

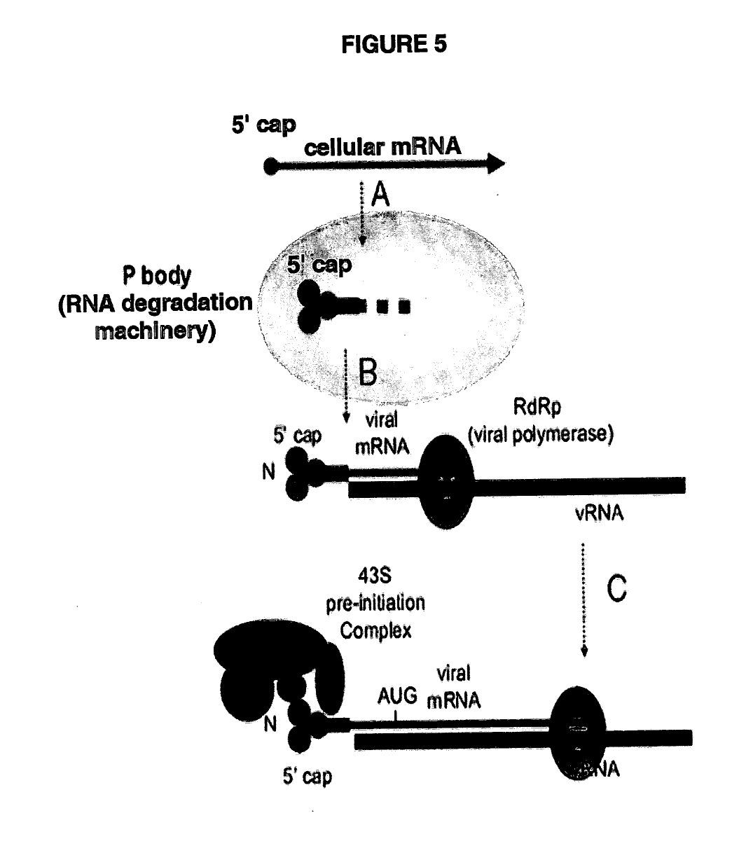

We recently found that N functions as a translation initiation factor by binding to the 5′ cap of viral mRNA, where it can replace the cellular cap binding complex, eIF4F, to mediate the early steps of viral mRNA translation ref. 34. An attractive possibility is that, during replication, N first binds to and protects cellular mRNA caps in P bodies and remains bound to the 5′ caps during transcription catalyzed by the RdRp. N would then be poised to serve in translation initiation immediately following viral mRNA synthesis (FIG. 5). In this regard, it is noteworthy that bunyaviral mRNA translation is coupled with transcription (i.e., translation initiates before viral mRNA is completed) (28). This may reflect efficient translation initiation by N on nascent viral mRNA. Several interesting facets of N-mediated cap snatching remain to be elucidated and are not included in this model. For example, it is unclear whether N associates with mRNA caps before localization to P bodies, or whether N migrates to P bodies and then binds to and protects 5′ caps. We think it more likely that N binds to 5′ caps before accumulating in P bodies, as prior association of N with 5′ caps might enable more efficient protection against a subsequent encounter with DCP2/1 in P bodies, and N is able to recognize capped oligonucleotides outside the context of P bodies (FIGS. 1A and B). Also, as alluded to earlier, it will be of great interest to verify whether the nuclease that generates the oligomeric cap primer is associated with the RdRp, and to understand the relationship between P body association and virus assembly and budding.

The nodavirus brome mosaic virus, which is a positive strand RNA virus, and the yeast retrotransposon Ty3, associate with P bodies during replication and transposition, respectively (29, 30). It is likely that additional viruses and virus-like elements associate with P bodies during their replication. In addition to associating with P bodies to sequester 5′ caps, the presence of hantavirus N in P bodies is likely indicative of further functions for P bodies in Bunyavirus replication. In particular, as N functions in the recognition of its tripartite genome, it is probable that encapsidation of viral RNA into capsids takes place in P bodies. This would potentially enable coordinated incorporation of the multipartite genome into assembling capsids.

Further details of FIG. 5 and the experiment(s) which yielded the data reflected in that figure are as follows.

### FIG. 5.

Turnover of cellular mRNA results in transport to P bodies, where viral N shelters the 5_termini from decapping and degradation (A). The viral RdRp uses the capped 5_termini during transcription initiation to generate nascent viral mRNA using the minus strand viral RNA template (B). N then recruits the 43S preinitiation complex during the process of translation initiation (C).

### Example 7

### N Facilitates Translation of Capped mRNA

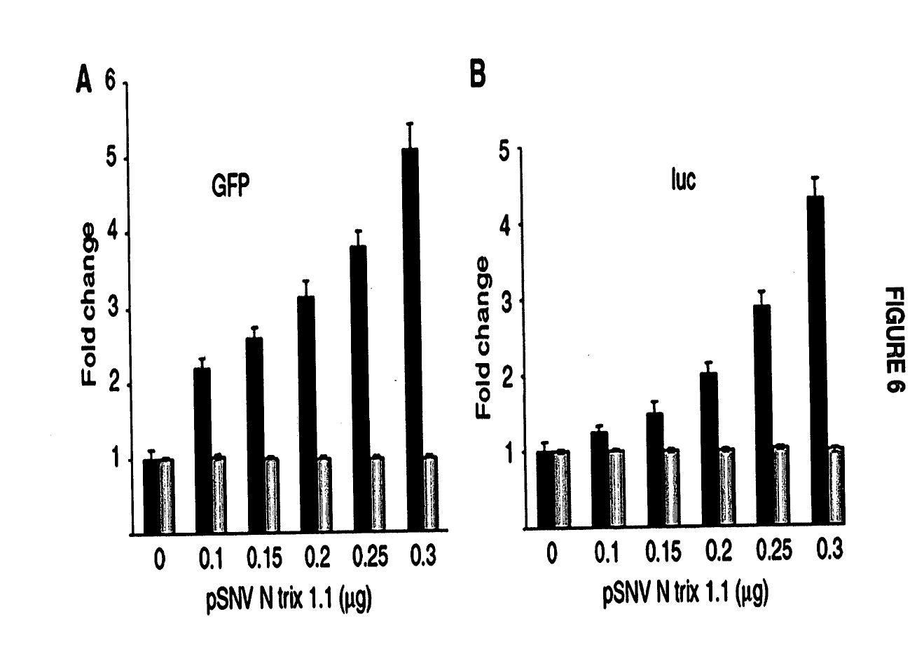

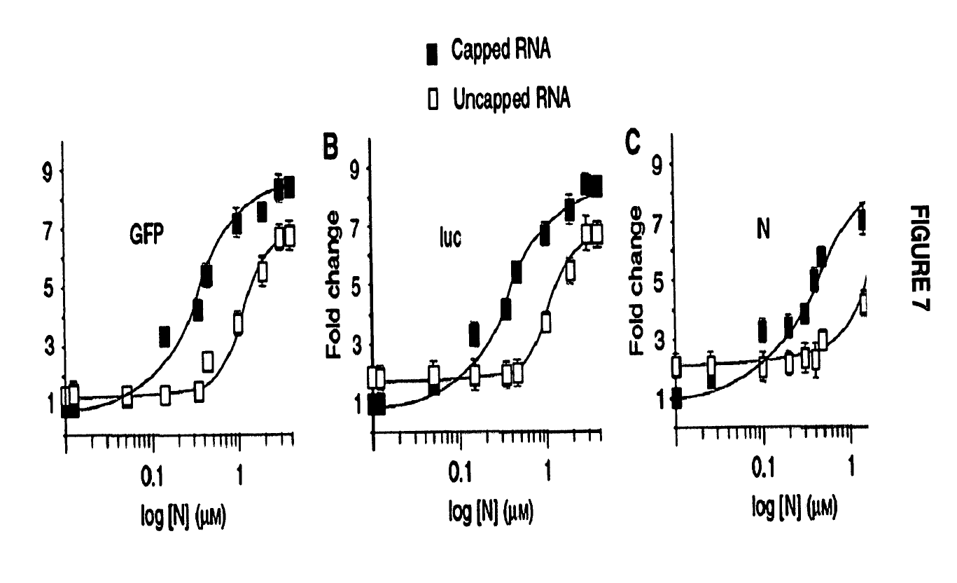

Co-expression of N with various reporter mRNAs yielded unexpected evidence, consistent with the idea that the steady state expression of reporter proteins was augmented by N. To examine this apparent N-dependent increase in protein expression, we co-transfected HeLa cells with increasing amounts of a plasmid that expresses Sin nombre hantavirus (SNV) N (or an empty expression vector) and a constant amount of a reporter plasmid expressing either green fluorescent protein (GFP) or luciferase (luc) mRNA. At 36 h after transfection, cells were harvested, and GFP expression was quantified by flow cytometry and luc was measured using a quantitative enzymatic assay. We observed a concomitant increase of about five-fold in both GFP expression and luc expression with increasing amounts of N expression plasmid (FIGS. 6A and 6B). Quantitative RT-PCR (real-time PCR) with primers corresponding to a segment in the centre of the mRNA indicated that N does not detectably affect intracellular amounts of either GFP or luciferase mRNA (FIGS. 7A and 7B), suggesting that N augments expression at the translational level.

We next used rabbit reticulocyte extracts to carry out in vitro translation reactions with reporter RNA containing or lacking a 5′ m7G cap. When increasing amounts of bacterially expressed purified N, were added translation of each of the three reporter mRNAs was enhanced. This effect of N was significantly more efficient and manifested at a lower N concentration when the reporter mRNA contained a 5′ m7G cap (FIG. 7A-C). It should be noted that the expression of N from the mRNA in FIG. 7C was used merely as a reporter, analogous to GFP or luc, and did not significantly contribute to the amount of N in the reaction.

Further details of FIGS. 6 and 7 and the experiment(s) which yielded the data reflected in those figures are as follows.

### FIG. 6.

HeLa cells were transfected with a constant amount of reporter plasmid and increasing amounts of a plasmid expressing hantavirus N. Evaluation of N expression on western blots with anti-N antibody indicated that N expression increased along with increasing amounts of plasmid, as expected (not shown). At 36 h after transfection, cells were harvested and GFP or luc expression was quantified, by flow cytometry or enzymatically, respectively (dark bars). (A) Expression of GFP is shown. (B) luc as a function of increasing N is shown. Steady-state GFP mRNA and luc mRNA were quantified using ‘real-time’ RT-PCR with primers specific for a segment in the centre of each reporter RNA. In both (A, B), the results of this latter analysis are depicted with light bars. FIG. 7.

We examined the effect of increasing N on the translational expression of three reporter mRNAs containing or lacking a 5′ cap using rabbit reticulocyte lysates. Capped and uncapped mRNA encoding GFP, luc or N were translated in vitro in the presence of 35S-methionine and increasing amounts of N in (A-C), respectively. Translation products were then electrophoresed on SDS polyacrylamide gels, and the amount of translation product was quantified by phosphorimage analysis. Translation of capped and uncapped RNA is depicted with filled and open squares, respectively. The amount of labelled protein synthesized with capped RNA in reactions lacking N was normalized to 1 (this cannot be indicated on the log scale). In the absence of N, expression of the indicator proteins was slightly higher with uncapped than capped RNA. This is consistent with earlier observations (Svitkin et al, 1996). Thus, the amount of expression at the lower concentrations of N is equivalent to background levels of expression from capped and uncapped RNA for each of the indicator RNAs.

### Example 8

### N Binds to 5′ Caps and Mediates Preferential Translation of Viral mRNA

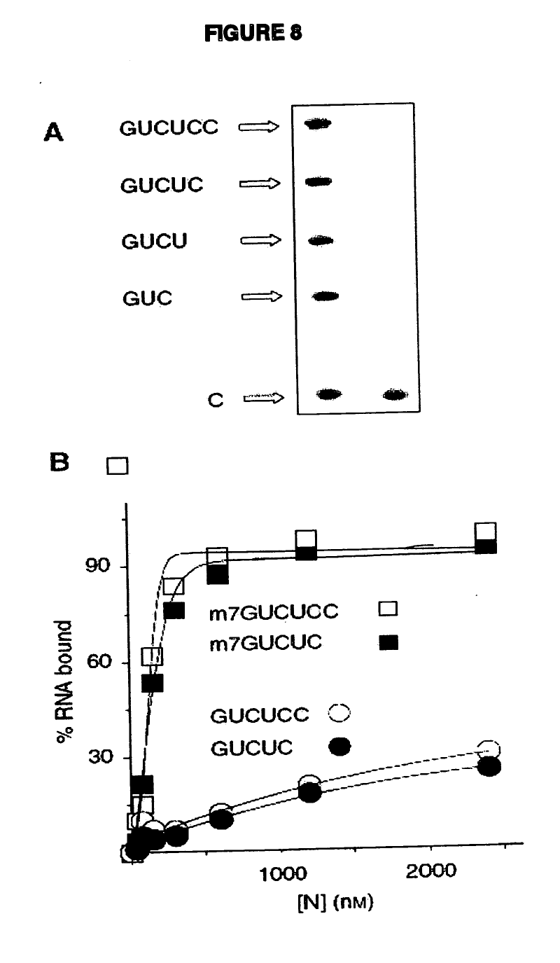

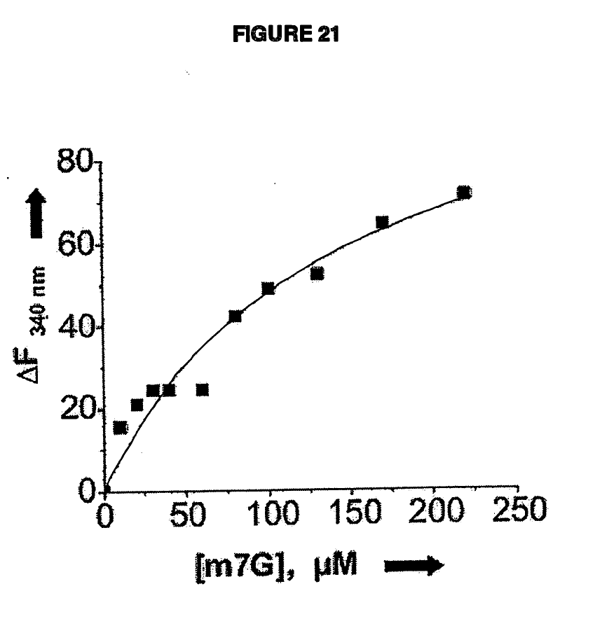

As N preferentially enhanced the translation of capped mRNAs, we next asked whether N interacts with the 5′ end of capped RNAs. We synthesized radioactively labeled capped and uncapped RNAs, 3-6 nt in length (FIG. 8A) and carried out filter binding studies with each of these short RNAs to assess binding by N. This indicated that N binds to capped but not uncapped penta- and hexanucleotide RNA at a Kd of 120-130 nM (FIG. 8B). However, there was no detectible binding with either capped or uncapped tri- and tetranucleotide RNA (FIG. 20). We also examined interaction between N and free cap using fluorescence spectroscopic analysis. The interaction of N with free cap is at least three orders of magnitude weaker than that observed for N with capped penta- or hexanucleotide RNA (FIG. 21). These data suggest that translation enhanced by N is superior for capped mRNAs owing simply to the ability of N to bind to capped 5′ ends.

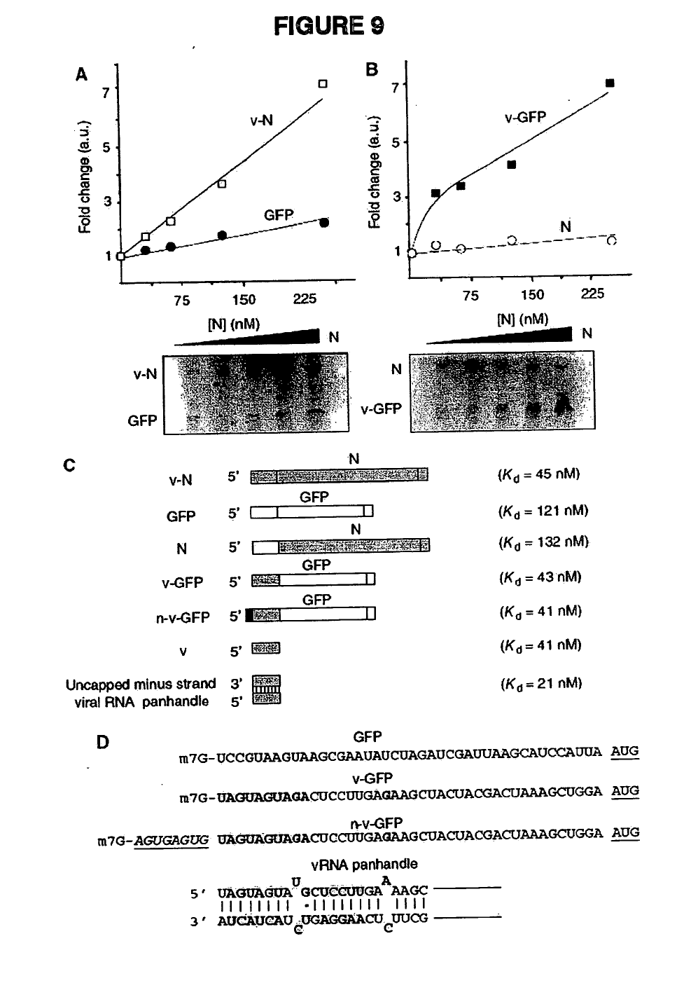

Hantaviruses do not abrogate general cellular mRNA translation. Nonetheless, if N enhances translation, viral mRNA might be preferentially translated relative to nonviral mRNA. The previously described ‘reporter RNA’ encoding N (FIG. 7C) contained the N gene but lacked this viral 5′ non-coding region, and N-mediated translation of this RNA was similar to that of the GFP and luc reporter mRNAs. As with all Bunyavirus, the 5′ ends of hantavirus mRNAs contain approximately 10 non-viral nucleotides that arise from capsnatching. The 5′ non-coding region from hantavirus S segment mRNA is 44 nt in length, not including non-viral nucleotides. We carried out a competitive assay to examine the translation of an mRNA containing the viral 5′ non-coding sequences relative to a second reporter (GFP) containing a non-viral leader of equal length. Equimolar amounts of these two capped RNAs were added together to reticulocyte extracts with increasing amounts of purified N. N-mediated enhancement of translation was superior for the viral mRNA when compared with the GFP RNA, yielding an increase in viral mRNA expression of about seven-fold (FIG. 9A). We generated two additional mRNAs in which the 44 nt leader regions were interchanged between the two reporter genes. Increasing amounts of N resulted in preferential expression of GFP from the chimaeric mRNA containing the capped viral 5′ leader (FIG. 9B). Thus, the translation of non-viral mRNA can be facilitated by N (FIG. 7), but in a competitive reaction containing both viral and non-viral mRNA translation of mRNA containing the 5′ non-coding sequences from the virus is robust when compared with mRNA harbouring a non-viral leader.

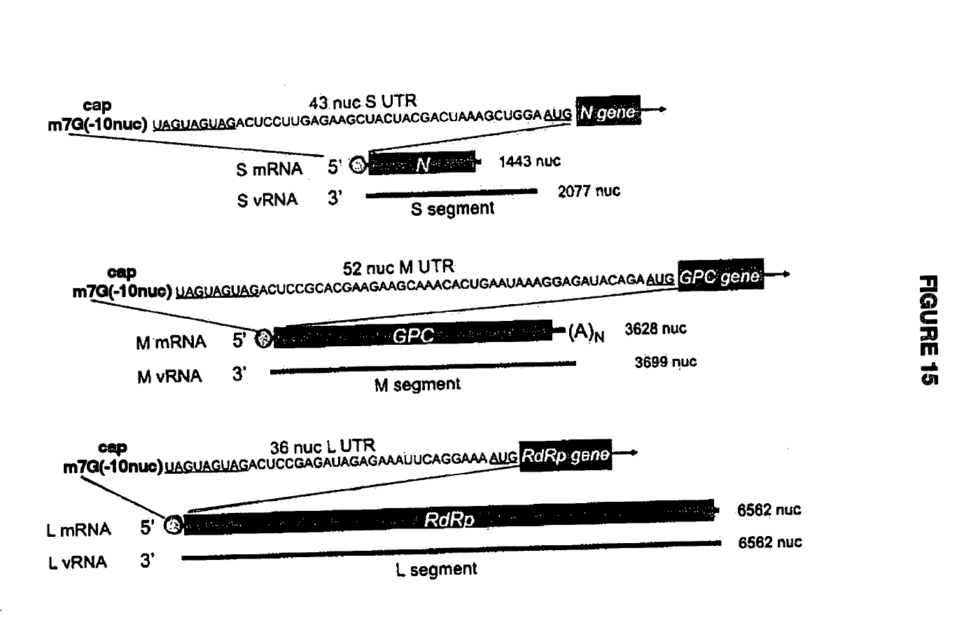

RNA-binding assays of N for each of these capped RNAs indicated that RNA containing the 5′ leader region from viral mRNA interacted with N at significantly higher affinity than RNA with a non-viral leader (FIG. 9C). Further, the viral leader region was sufficient for higher affinity binding by N. Preferential translation of viral mRNA, and high-affinity binding by N, is not diminished by the presence of capped non-viral nucleotides at the 5′ end, as would be present on bona fide viral mRNA (FIG. 9C) (Mir and Panganiban, submitted). See also FIGS. 15 through 18.

Further details of FIGS. 8, 9, 20, 21, 15 and 16 and the experiment(s) which yielded the data reflected in those figures are as follows.

### FIG. 8.

(A) Short radioactively labelled capped and uncapped RNAs were synthesized using T7 RNA polymerase and a-32P-CTP. As the third nucleotide of transcription products arising from the T7 promoter is the first C residue of the RNA, only molecules 3 nt long or greater were labelled. These short RNAs were separated on, and recovered from, a high percentage denaturing polyacrylamide gel. The image depicts such a gel. The leftward lane displays the series of short oligoribonucleotides arising from transcription. The rightward lane contains unincorporated CTP as a migration control. (B) Each RNA was incubated with increasing concentrations of purified N, and association of RNA with N was quantified by filter binding. Binding of N with capped (m7GUCUCC) or uncapped (GUCUCC) are indicated with open squares and circles, respectively, and with m7GUCUC and GUCUC with closed squares and circles, respectively. Binding experiments carried out with both capped and uncapped RNAs less than 5 nt in length exhibited negligible binding with N. For example, binding with the 4-nt long RNA, GUCU, in capped and uncapped form is shown in FIG. 20.

### FIG. 9.

(A) Equimolar amounts of capped mRNA containing the 5′ untranslated region from S segment mRNA and encoding N (v-N), and an mRNA containing a non-viral leader region and encoding GFP (GFP) were added together to reticulocyte extracts containing increasing concentrations of N as indicated. The concentration of each mRNA was approximately 45 nM. Labelled N and GFP were separated by PAGE and quantified by phosphorimage analysis (shown below the graph). Similar results were obtained in three separate experiments. In (B), the leader regions from the mRNAs of (A) were interchanged. Thus, one mRNA contained the 5′ viral UTR preceding the GFP gene (v-GFP), and a second mRNA contained the non-viral leader preceding the N gene (N). Translation and quantitation were as in (A). (C) Radioactively labelled capped RNAs were used in binding reactions with purified N and the binding affinity (Kd) was determined for each. Viral sequences are shown schematically in grey, whereas non-viral sequences are in white. n-v-GFP contains a 9 nt non-viral cap simulating cellular RNA derived from cap-snatching (shown in black). Note: The leader regions are not shown to scale relative to the N and GFP genes. The untranslated leaders, GFP gene, and N gene are 43, 798, and 1287 nt in length, respectively. (D) Comparison of the leader sequences from GFP, v-GFP, and n-v-GFP mRNA and minus strand S segment viral RNA. Nucleotides required for high-affinity binding to the vRNA panhandle are depicted with shading and include nucleotides from both the 5′ and 30 termini (Mir and Panganiban, 2005). The 5′ terminal nucleotides of +strand mRNA required for binding by N is also indicated by shading. As the termini of the viral genome segments consist of imperfect inverted repeats, the 5′ sequences of both plus and minus strand viral RNA are similar. Nucleotide differences in the 5′ sequence of mRNA relative to the 5′ sequence of minus strand vRNA are indicated with bold lettering. Leader sequences of v-GFP and n-v-GFP. The 9-nt-long non-viral leader of n-v-GFP, and the start codon of the mRNAs are underlined.

### FIG. 15

The nucleotide sequences of the 5′ untranslated regions of three hantavirus genome segments are shown in the figure. In addition, the relative positions of the truncated viral mRNAs are shown schematically in alignment with each of their corresponding genome segments.

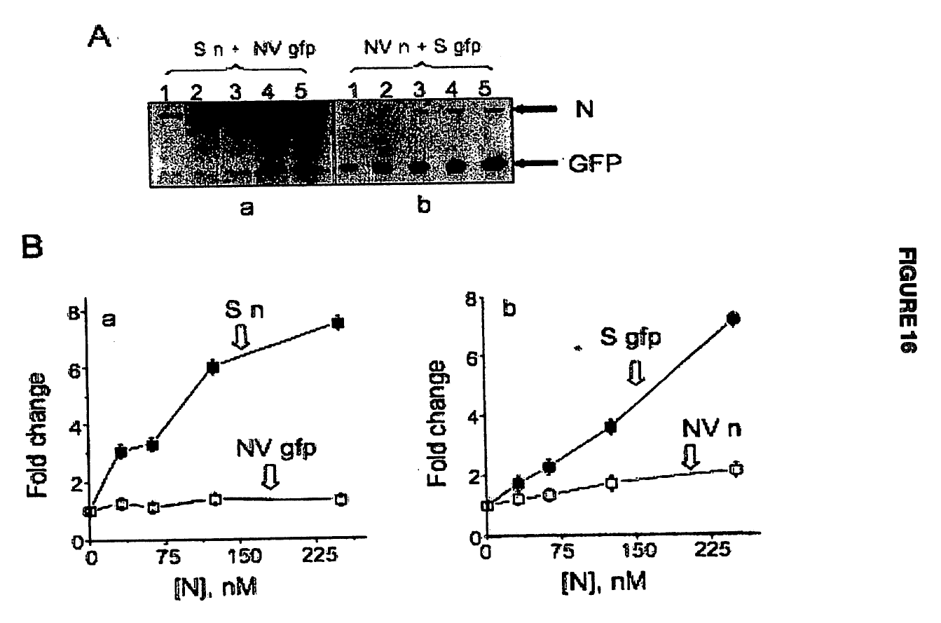

### FIG. 16.

S n is an mRNA derived from the negative sense SNV S segment. It has 5′ untranslated region (UTR) of that mRNA, which is 42 nucleotides long, with the n gene. NV n contains a nonviral UTR that is 42 nucleotides long, with the n gene. S gfp and NV gfp are mRNAs with either a 5′ UTR derived from the SNV S segment mRNA or the same 42 nucleotide non viral sequence as in NV n. However, both S gfp and NV gfp contain the gfp gene rather than the n gene. These mRNA's were synthesized by in vitro T7 transcription as described in Examples 7-10 Materials and Methods sections. In competitive translation assays two mRNAs are translated together in rabbit reticulocyte lysates at different input concentrations of bacterially expressed and purified N protein, radioactively labeled with S35 methionine, fractionated by SDSPAGE, and translation products quantified by phosphorimage analysis. Panel A depicts first the competitive translation of mRNA molecules S n and NV gfp and then S gfp and NV n. N and GFP are indicated by arrows. The concentration of input N was increased from 0 nM (lane 1), 31 nM (lane 2), 62 nM (lane 3), 125 nM (lane 4) to 250 nM (lane 5). Panels B and C are graphical representations of the N and GFP expression following phosphorimage analysis. The normalized intensity of N and GFP was plotted at different input concentration of bacterially expressed and purified SNV N.

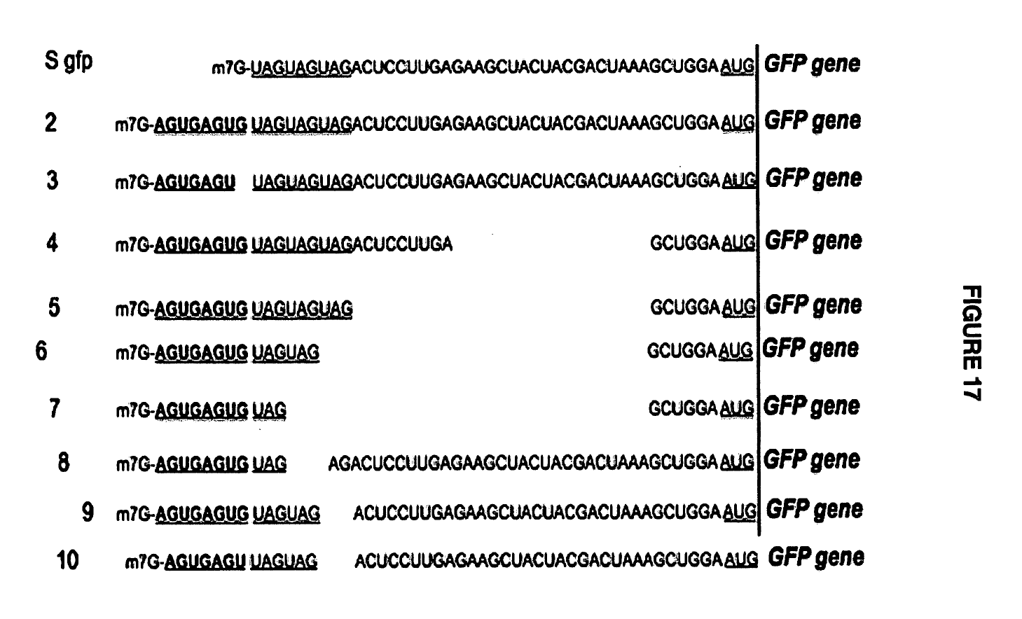

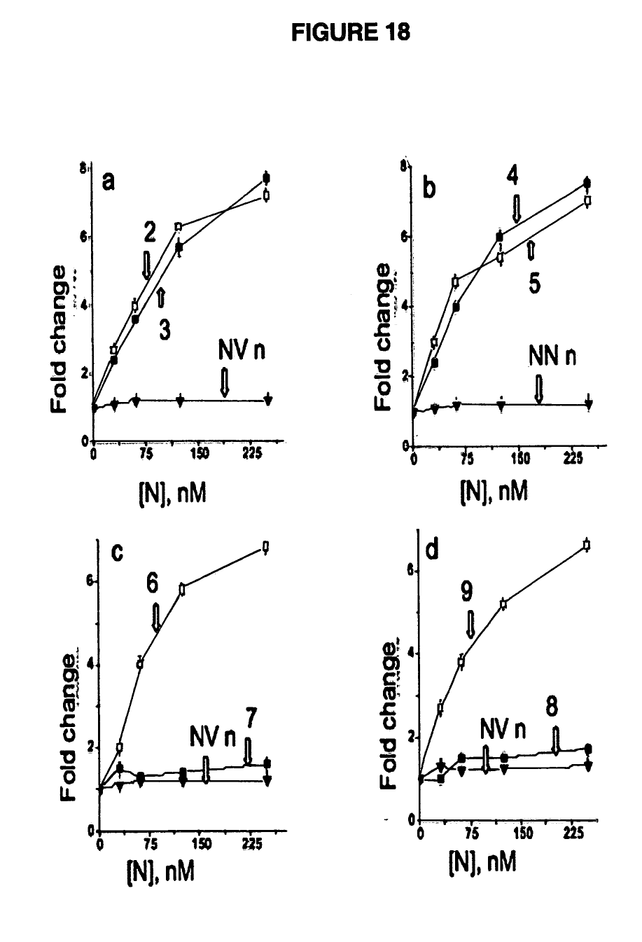

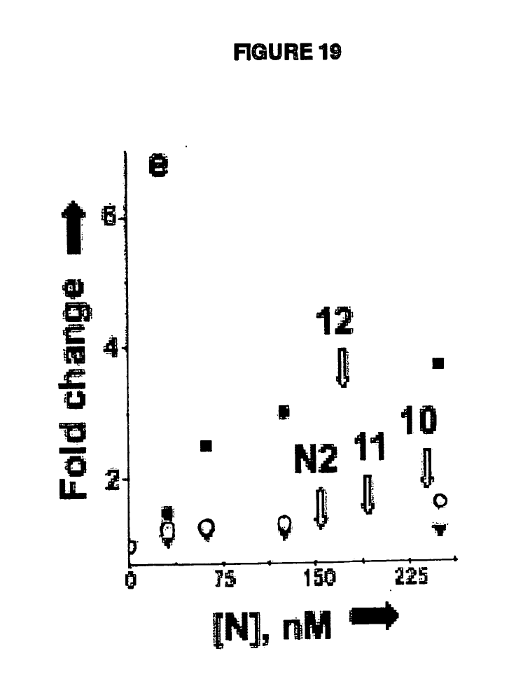

FIG. 17. mRNAs containing deletions in the viral UTR. The top sequence shows the nucleotides derived from the viral RNA template. Viral mRNAs contain heterologous 5′ caps derived from the process of cap-snatching. Thus, the remainder of the mRNAs contain an arbitrary nine nucleotide nonviral sequence appended to the 5′ end of the templated sequence to mimic the presence of a nonviral 5′ cap acquired from cellular mRNAs by cap-snatching. mRNAs devoid of various nucleotides in the viral UTR are depicted below the complete sequence.
FIGS. 18 and 19. Competition translation analysis of some of the deletion derivatives shown in FIG. 17. In each case, the various mRNAs lacking portions of the UTR were added to translation reactions along with competitor mRNA containing a nonviral UTR and the n gene. Numbers denote the specific mRNAs displayed in FIG. 17.

### FIG. 20.

Binding of capped and uncapped tetrameric RNA with N. RNA filter binding was carried out as described in FIG. 8. Capped RNA-open squares. Uncapped RNA-closed triangles.

### FIG. 21.

A constant amount of N (150 nM) was incubated with increasing concentrations of m7G and the fluorescence spectrum of N (300 nm-450 nm) was recorded at each input concentration of the cap (m7G). Since m7G also yields an independent fluorescence signal in this wavelength range, the fluorescence spectrum of free m7G at each input concentration was also recorded. The cap-dependent change in N fluorescence (delta F) was calculated by subtracting the fluorescence value of N at 340 nm in the absence of cap from the fluorescence values at different input concentrations of cap.

### Example 9

### N Stably Binds to the 43S Pre-Initiation Complex and Replaces eIF4G

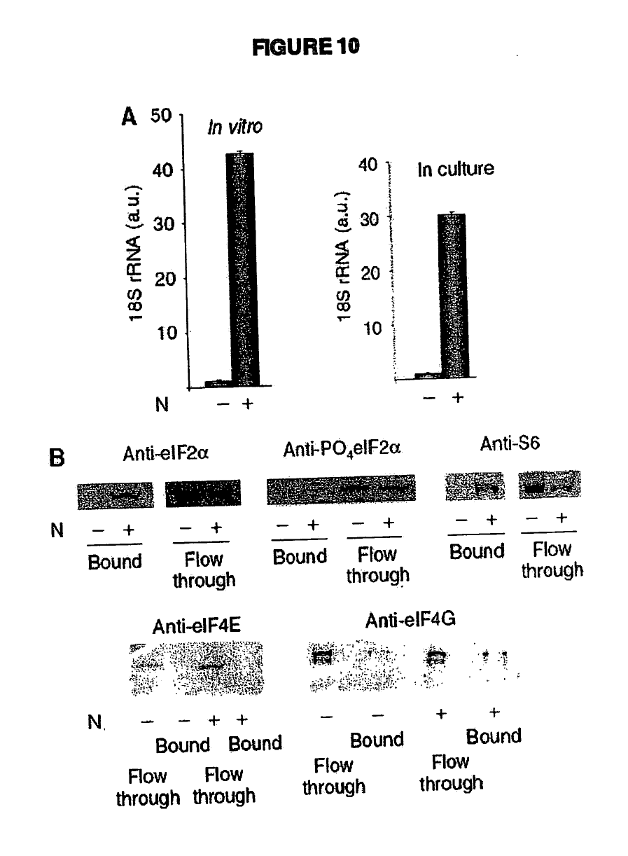

eIF4F binds to mRNA by way of eIF4E, and to the eIF3 and the 43S pre-initiation complex by way of eIF4G. To determine whether N may interact with the 43S pre-initiation complex, we added his6-tagged N to rabbit reticulocyte lysates, recovered N using Ni-NTA beads and used a quantitative assay for 18S rRNA to quantify 40S ribosomal subunits associated with N. These assays indicated that 18S rRNA was associated with N (FIG. 10A), suggesting that N interacts with 40S eukaryotic ribosomal subunit. We carried out a similar experiment with lysates derived from 293 cells expressing his6-tagged N following transfection with an N expression construct. Again we observed that 18S rRNA was recovered with N on Ni-NTA beads, indicating that N interacts directly or indirectly with the 43S pre-initiation complex in vivo (FIG. 10A).

In the unphosphorylated form, eIF2a is a functional component of the 43S pre-initiation complex, whereas phosphorylated eIF2a is not associated with the pre-initiation complex. We determined whether eIF2a co-purified with his6-tagged N from lysates of transfected cells on Ni-NTA columns using western blot analysis with antibody specific for the unphosphorylated and phosphorylated forms of eIF2a. This indicated that unphosphorylated eIF2a but not phosphorylated eIF2a was associated with N (FIG. 10B). Moreover, western blot analysis indicated that S6 ribosomal protein also co-purified with N (FIG. 10B). These data again suggest that N interacts with the 43S pre-initiation complex. In contrast, components of the eIF4 cap-binding complex, eIF4E and eIF4G, did not co-purify with N in parallel western blots (FIG. 10B). Thus, association of the 43S pre-initiation complex with N does not require the eIF4F cap-binding complex.

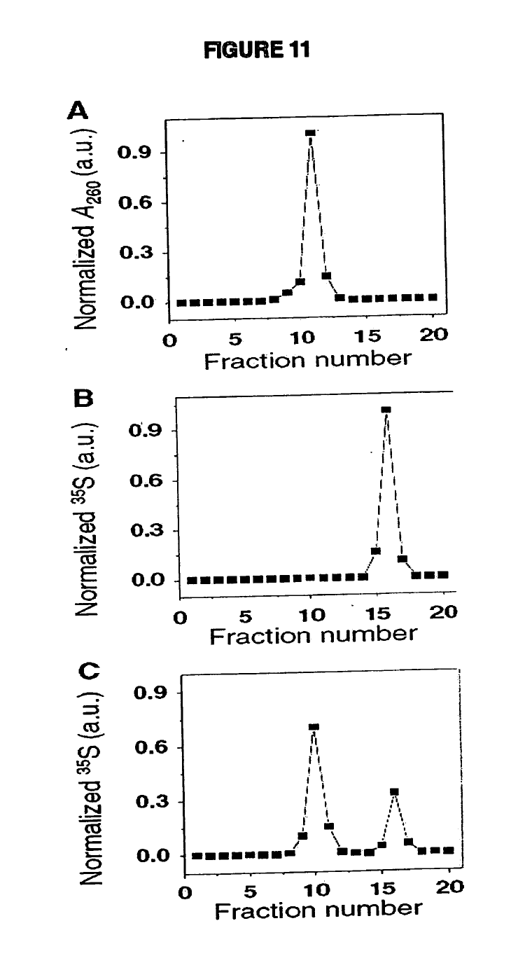

To verify that interaction between N and the 43S preinitiation complex was mediated by way of direct interaction rather than indirectly through an mRNA bridge, we dissociated ribosomes into large and small subunits by incubation with puromycin, purified 40S small ribosomal subunits by sucrose gradient centrifugation, and asked whether N could interact directly with purified 40S subunits. The purified 40S subunits were resedimented, detected by monitoring optical density and yielded a sedimentation profile indicative of a homogeneous 40S preparation (FIG. 11A). We synthesized ³⁵S-labelled N in reticulocyte extracts, and purified the labelled protein by denaturation, recovery on Ni-NTA columns, and renaturation. Sedimentation analysis of this purified ³⁵S-labelled N protein indicated that N migrated to a distinct position high in the gradient (FIG. 11B). Significantly, incubation of N with purified 40S subunits resulted in co-migration of N with small subunits indicative of interaction between N and the 40S subunit (FIG. 11C). A radioactively labelled control protein (GFP) did not interact with 40S ribosomal subunits and remained near the top of the gradient (data not shown). These data indicate that N binds directly to a component of the small ribosomal subunit and that association is not through an mRNA bridge. However, the data do not unequivocally distinguish between whether N interacts directly with the 40S subunit or with residual eIF3 bound to the 40S subunit.

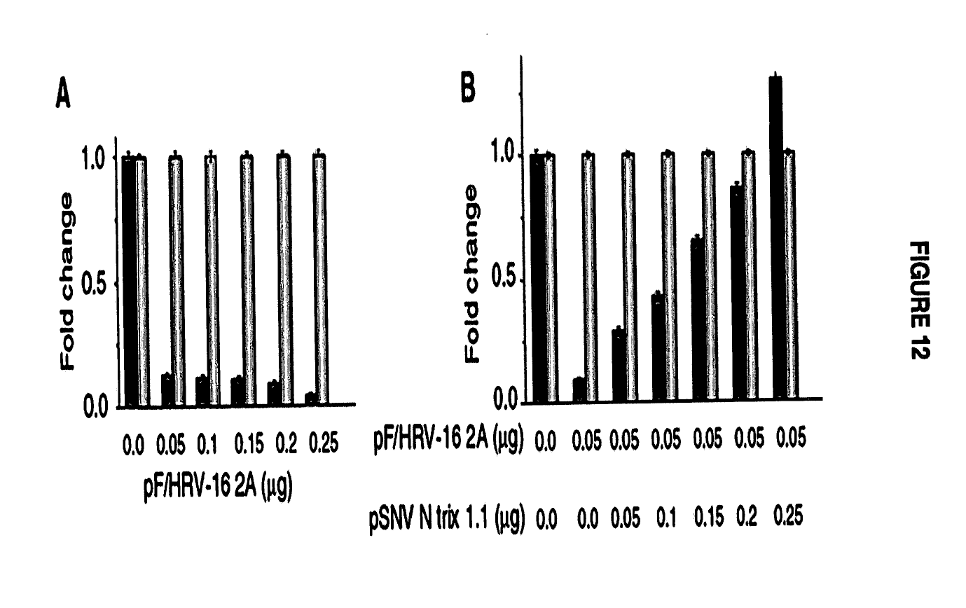

We next asked whether N is likely to functionally replace eIF4G. Some members of the picornavirus family shut off host mRNA translation through proteolytic cleavage of eIF4G, a process mediated by the viral 2A protease (Etchison et al, 1982; Liebig et al, 1993; Haghighat et al, 1996). We cotransfected cells with a plasmid expressing GFP reporter mRNA along with increasing amounts of a plasmid that expresses the 2A protease of human rhinovirus 16 (HRV-16). Although the 2A cleavage products of eIF4G sometimes retain residual activity (Ali et al, 2001), as expected, the presence of this plasmid expressing 2A protease dramatically reduced translational expression of the GFP reporter mRNA (FIG. 12A). Significantly, co-expression of N overcame this translational inhibition resulting from the 2A-expressing plasmid (FIG. 12B). Thus, N-mediated translation initiation appears to take place under conditions where eIF4G is proteolytically inactivated. Taken together, all these data are consistent with a simple model where N supplants eIF4G in bridging to the 43S pre-initiation complex.

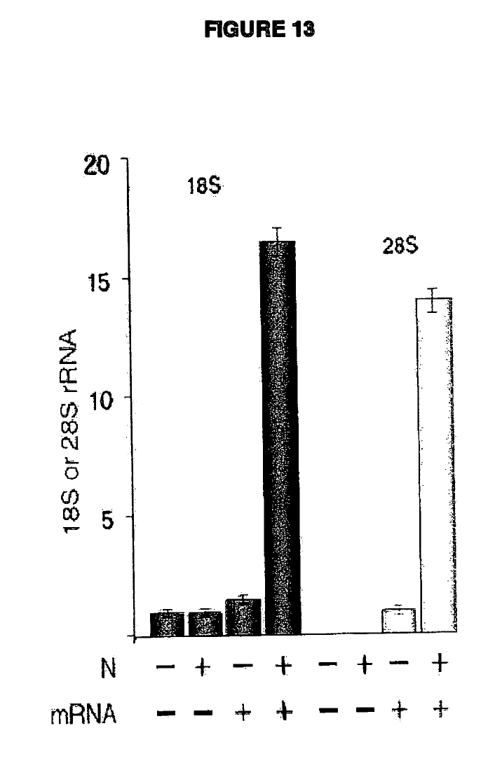

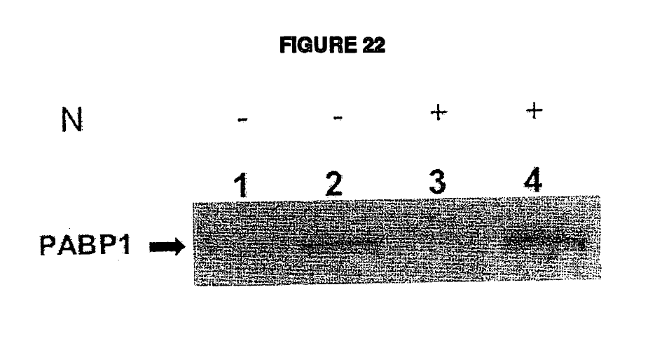

We used a ribosome-loading assay to see whether N can facilitate loading of small ribosomal subunits onto the 5′ end of mRNA. A 415-nt-long mRNA containing a 15-nt-long poly A tail and 200-nt-long non-coding 5′ leader sequence was incubated in rabbit reticulocyte lysates in the presence or absence of N. This mRNA was then purified from the reaction mixture by virtue of its poly A tail using poly-dT sepharose beads. Ribosomes loaded onto the isolated mRNA were then quantified by measuring 18S and 28S rRNA associated with the isolated mRNA. We observed a significant increase in both 18S and 28S rRNA when both N and mRNA were present in rabbit reticulocyte lysates indicative of enhanced ribosome loading onto the isolated mRNA (FIG. 13). As the recruitment of the 43S pre-initiation complex to the 5′ end of mRNAs is conventionally considered to be the rate-limiting step for translation, these data suggest that N increases the rate of recruitment of the 43S pre-initiation complex onto mRNAs. Cellular mRNA is circularized through association between eIF4G in the eIF4F cap-binding complex at the 5′ end and poly A-binding protein (PABP) at the 30 end leading to more efficient translation (Tarun and Sachs, 1996; Gray et al, 2000). As eIF4G appeared to be dispensable for N-mediated translation initiation, we asked whether N may interact with PABP to effect circularization. We carried out co-precipitation experiments in which his6-tagged N from lysates of transfected cells was recovered on Ni-NTA beads and the recovered material was analysed by western blot analysis with anti-PABP antibody. On the basis of this approach, there was not detectible stable association between N and PABP (FIG. 22).

Further details of FIGS. 10-13 and 22 and the experiment(s) which yielded the data reflected in those figures are as follows.

### FIG. 10.

FIG. 10 illustrates that N interacts with the pre-initiation complex. (A) N was incubated with rabbit reticulocyte lysates and recovered with Ni-NTA beads. The bound material was eluted from the Ni-NTA, RNA was purified, and 18S rRNA was quantified using real-time RT-PCR. The leftward graph depicts the relative amount of 18S rRNA associated with Ni-NTA beads in the absence and presence of N. The rightward graph depicts an analogous experiment carried out with 293 cells that were transfected with either an N-expressing plasmid or its parental vector, as a negative control. N was recovered from the lysates of transfected cells using Ni-NTA and 18S rRNA that co-purified with N quantified by real-time RT-PCR. (B) A set of western blots to examine the association of peptide constituents of the 43S pre-initiation complex, and the eIF4F capbinding complex, that co-purify with N. N was expressed by transfection, isolated from the lysates of these cells using Ni-NTA columns, bound material was recovered and subjected to western blot analysis with primary antibodies as indicated. Peptides that copurify with N (bound), or that flow through the column are indicated.

### FIG. 11.

FIG. 11 shows that N binds directly to the small ribosomal subunit. 40S small ribosomal subunits were prepared by incubation of ribosomes in the presence of puromycin and purified from large ribosomal subunits and mRNA. (A) Purified 40S subunits were then resedimented on a sucrose gradient. (B) N protein was expressed by in vitro translation in the presence of 35S-methionine, purified from the translation mixture by denaturation with urea, recovery on Ni-NTA beads, renaturation by dialysis and sedimented in parallel with 40S subunits. (C) N was incubated with excess purified 40S subunits prior to sedimentation. Leftward fractions correspond to those from the bottom of the gradient.

### FIG. 12.

FIG. 12 illustrates that N replaces eIF4G. (A) HeLa cells were co-transfected with a plasmid expressing reporter GFP, along with increasing amounts of pF/HRV-16 2A, which expresses the 2A protease of HRV-16. GFP expression was quantified using flow cytometry as in FIG. 1 (dark bars) and GFP mRNA was quantified using real time PCR (light bars). (B) Cells were transfected with a constant amount of GFP expression plasmid, a constant amount of 2A expression plasmid (0.05 ug) sufficient for significantly reducing translation of the reporter gene, and increasing amounts of an N expression plasmid. GFP expression (dark bars) and steady state GFP mRNA (light bars) was quantified as in (A). In the experiments of both panels (A) and (B) the total amount of DNA used in the transfections was held constant by addition of parental vector.

### FIG. 13.

FIG. 13 illustrates that N promotes ribosome loading. A synthetic mRNA containing 30 poly A was incubated in reticulocyte lysates to allow translation. The synthetic polyadenylated RNA was recovered from the translation mixture using oligo dT beads. Ribosomes associated with the polyadenylated RNA were quantified by real-time RT-PCR with primer sets specific for 18 and 28S rRNA.

### FIG. 22.

N does not detectably associate with PABP. Cells were transfected with an N expression plasmid containing a his8 tag. N was recovered from cell lysates using Ni-NTA columns and PABP was detected by Western analysis with anti-PABP. Lanes 1 and 3 depict PABP associated with Ni-NTA columns in the absence or presence of N, respectively. Lanes 2 and 4 show PABP in the flowthrough from Ni-NTA columns in the absence or presence of N, respectively.

### Example 10

### N Replaces eIF4A

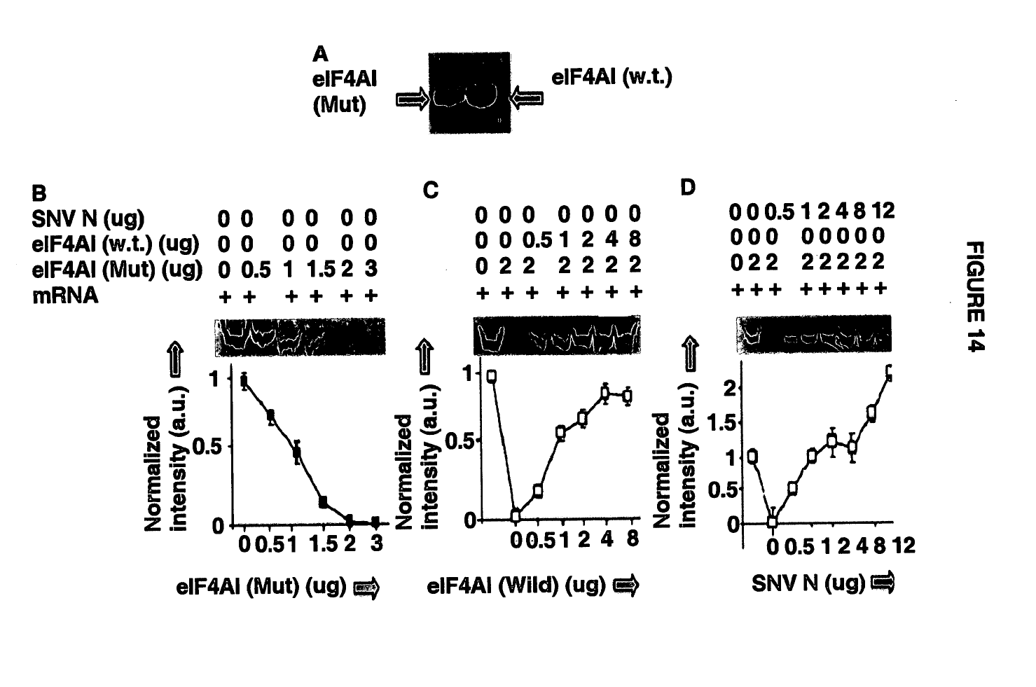

We next wanted to see whether N replaces the activity of the third constituent of the eIF4F complex, eIF4A. eIF4A is a DEAD box RNA helicase required for eIF4F function during cap binding and has been postulated to function in the scanning of the pre-initiation complex to the AUG start codon (Rogers et al, 2002; Hernandez and Vazquez-Pianzola, 2005). In comparison, N has an intrinsic ATP independent activity that facilitates transient RNA duplex dissociation (Mir and Panganiban, 2006). eIF4A migrates on and off the eIF4F complex, where it functions in concert with eIF4E and eIF4G, and this interconversion between the complexed and free forms of eIF4A appears to be necessary for eIF4A function. We used a dominant-negative mutant of eIF4A defective in cycling through eIF4F complex, which dramatically inhibits translation in rabbit reticulocyte lysates, to see whether N mediates translation initiation in an eIF4F independent manner (Pause et al, 1994). Wild-type and dominant-negative eIF4A were expressed in bacteria and purified (FIG. 14A).

Consistent with published characterization of this dominant-negative eIF4A protein (Pause et al, 1994), 2 mg of the dominant-negative mutant protein inhibited translation of a reporter mRNA in rabbit reticulocyte lysates by about 98% (FIG. 9B), and translation could be significantly rescued by the addition of 2-4 mg of wild-type eIF4A when the dominant-negative protein was present (FIG. 14C). Notably, translation inhibited by dominant negative eIF4A could also be completely overcome by adding 1.5 mg of N protein to the translation reaction (FIG. 14D). These data indicate that that N functionally substitutes for eIF4A, and together with the earlier presented data indicate that N functionally substitutes for the entire eIF4F cap-binding complex.

Further details of FIG. 14, and the experiment(s) which yielded the data reflected in that figure, are as follows.

### FIG. 14.

FIG. 14 shows that N functionally replaces eIF4A. (A) Bacterially expressed and purified wild-type and mutant eIF4A were used in in vitro translation reactions containing luciferase mRNA. (B) Effect of dominant-negative mutant eIF4A on translation. Translation was quantified by SDS-PAGE followed by phosphorimage analysis of radioactively labelled luc. (C) Similar reactions were carried out in the presence of fixed amount (2 mg) of mutant eIF4A and increasing amounts of wild-type eIF4A. (D) Translation reactions in the presence of 2 mg of mutant eIF-4A and increasing amounts of N.

### Additional Analysis of the Experiments of Examples 7-10

### N as a Translation Initiation Factor.

N-mediated translation initiation is a viral strategy that is the complement to the use of an IRES. The latter tactic is employed by the picornaviruses and some flaviviruses. Although an IRES is a cis-acting element that functionally supplants the requirement for cap-dependent translation, N is a trans-acting element that replaces eIF4F.

It is likely that N mediates initiation through a simple mechanism. There is no overt similarity between N and the eIF4F components. Nonetheless, the three-dimensional structure of cellular cap-binding peptides human eIF4E includes two W residues (W52 and W102) that hold the guanine residue of a cap-analogue through stacking interactions. An acidic residue (E103) further stabilizes this association (Marcotrigiano et al, 1997; Matsuo et al, 1997). There may be weak alignment of this region with a segment of N of identical length (a.a. 119-166) containing appropriately spaced W119 and Y165E166 residues. Binding of eIF4G with the eIF3 and the 43S pre-initiation complex is mediated by a portion of the central region of eIF4G (Korneeva et al, 2000; Schutz et al, 2008). ClustalW comparison of N with this region of eIF4G indicates weak alignment with the amino-terminus of N. These regions of both peptides contain antiparallel alpha-helices, in coiled-coil (N) or in HEAT (eIF4G) configuration (Marcotrigiano et al, 2001; Boudko et al, 2007). However, interaction of N with 40S subunits is likely mediated through a domain dissimilar to that responsible for eIF3 recognition by eIF4G. Finally, there is no apparent similarity between eIF4A and N. This may be expected as eIF4A is an ATP-dependent DEAD box helicase, N is an ATP-independent RNA chaperone.

N augments translation of both viral and non-viral mRNA. However, viral mRNA is recognized at higher affinity by N, and translation of viral mRNA is more robust in competitive in vitro translation reactions with non-viral mRNA. mRNA from all minus strand segmented RNA viruses is initiated with nucleotides acquired from the 5′ ends of cellular mRNA by cap snatching. Nonetheless, the motif in viral mRNA preferentially recognized by N is situated in the viral 5′ UTR and not affected by the presence of a short non-viral cap. Mutational analysis of the viral UTR indicates that the motif recognized by N for preferential translation is less than 10 nt in length (FIG. 9D) (Mir and Panganiban, submitted). In contrast, the motif in minus strand vRNA recognized at high affinity, which ostensibly initiates genome encapsidation, is the predominantly double-stranded viral panhandle formed by the juxtaposition of the 5′ and 30 vRNA termini (FIG. 9D) (Mir and Panganiban, 2004). The panhandle is recognized at higher affinity than viral mRNA. During replication, viral mRNA synthesis precedes and overlaps with genome replication. It will be of interest to see common RNA binding domains of N function in both processes.

It is likely that the general strategy of encoding a transacting factor to ensure efficient viral translation is not restricted to hantaviruses. It is probable that the N peptides of members of the other genera of the bunyavirus family, and perhaps the members of diverse families of segmented and non-segmented minus strand RNA viruses, also supplant the eIF4F complex. Several observations hint that RNA viruses may use this general scheme. Subgenomic Sindbis virus mRNA is translated when eIF4G is inactivated (Castello et al, 2006). Vesicular stomatitis virus impairs eIF4E function through dephosphorylation but sustain translation of its own mRNAs (Connor and Lyles, 2002). Similarly, influenza mRNA translation can occur when eIF4E is impaired (Burgui et al, 2007). Expression of Sendai virus N is required for the expression of a reporter gene from a viral vector (Wiegand et al, 2007). This could be due to an effect on transcription, as suggested by the authors, but their data are also consistent with a positive role of SeV N in translation.

### Implications for Viral Transcription.

The Bunyaviruses are unique among the negative-stranded RNA viruses in that transcription requires concomitant translation of the nascent viral mRNA (Bellocq and Kolakofsky, 1987; Barr, 2007). Coupling of transcription with translation appears to be necessary for successful RNA elongation by the RdRp through spurious premature transcription termination signals. Mechanistically, this may occur as ribosomes trailing the RdRp block the formation of higher order structures in the nascent RNA that function as inappropriate termination sites. N may promote more efficient loading of ribosomes onto nascent viral mRNA leading to higher ribosome density and ensuring mRNA elongation.

Apparent Lack of Circularization of Hantavirus mRNA.

Circularization of cellular mRNA mediated by the interaction between eIF4G and PABP enhances translation efficiency (Tarun and Sachs, 1996; Gray et al, 2000). However, circularization is probably not required for efficient N-mediated translation of viral mRNA, or else occurs through unidentified factors. Initiation can take place independently of eIF4F and we were unable to detect association between N and PABP. Moreover, of the three hantavirus mRNAs only that encoding the viral envelope protein is polyadenylated, whereas mRNA encoding N and the RdRp are not (Hutchinson et al, 1996).

It is worthwhile to contrast N-mediated translation initiation with viral peptides that associate with eIF4F. The potyviruses, a set of plant viruses related to the picornaviruses, encode a VPg that is attached to the 5′ end of the genome and that also operationally associates with eIF4E, potentially enabling functional circularization of the genome (Kang et al, 2005). Rotavirus NSP3A and Alfalfa mosaic virus (AMV) coat protein associate with both the 3′ end of their respective genomes and with eIF4G (Piron et al, 1998; Bol, 2005). This association with the eIF4F complex also enables genome circularization as the 30 termini of these viral genomes are not polyadenylated. Thus, NSP3A and AMV coat function as surrogates for PABP. For these viruses, interaction with the eIF4F complex is required for efficient replication.

There are a remarkable number of activities and functions associated with hantavirus N. These include its structural role as the capsid protein, its role as the principle player mediating the encapsidation of vRNA, its role as an RNA chaperone capable of reconfiguring the higher order structure of RNA, its role in genome replication in coordination with the viral polymerase, and now an unexpected role as a translational initiation factor with multiple complementary activities dedicated to that function. At the same time, N-mediated translation of viral mRNA can probably be considered to be a narrowly focused function ensuring efficient production of the viral peptides. In this regard, translation initiation by N would not be accompanied by the elegant and subtle regulatory capacity of the multi-component cellular translational complex.

### Materials and Methods

### Examples 1-6

### Filter Binding Studies

We examined the interaction of SNV N protein with capped or uncapped RNA or the 5′ terminal 10 nucleotides by synthesizing RNA from pTriEx. Transcription was by generated in T7 transcription reactions in the presence of radiolabeled P32 CTP. Transcription reactions contained 7 mM nucleotides. To generate capped RNA, the GTP concentration was reduced to 0.3 mM and 6 mM m7-GTP was added. Reactions for synthesis of the decamer RNA used in competition experiments (FIG. 1C) lacked GTP and contained either 7 mM m7 or 2′-O-methyl GTP. All binding reactions were carried out in RNA binding buffer (31) at a constant concentration of RNA with increasing concentration of N.

Reaction mixtures were incubated at room temperature for 30 to 45 min and filtered through nitrocellulose membranes under vacuum. Filters were washed with 10 ml of RNA binding buffer and dried. The amount of RNA retained on the filter at different input concentrations of N was measured using a scintillation counter. Data points were fit to a hyperbolic equation using the program Origin 6 (Microcal). Dissociation constants corresponding to the concentration of N protein required to obtain the half saturation in the binding profile were calculated, assuming that the complex formation obeys a simple bimolecular equilibrium.

We assumed that the plateau in the binding profile represents complete binding of RNA to allow the calculation at half saturation.

### Oligonucleotides, Enzymes, and Reagents.

PCR primers were from Sigma. All restriction enzymes were from New England Biolabs, Proof pro DNA polymerase was from Gene Choice, DNase I was from Invitrogen, and T7 transcription reagents were from Fermentase or Promega. 5′ mRNA cap analog was from Promega. P32 CTP was from Perkin-Elmer. All RNA purification kits were from Qiagen. Real-time PCR reagents including Power SYBR Green PCR Master Mix, MicroAmp 96-well plates, and optical adhesive covers were from Applied Biosystems. Reagents for confocal microscopy including cover slips, glass slides, and mounting medium was from BD Biosciences. All other chemicals were purchased from Sigma. All antibodies were from Abcam.

### Plasmids.

As reported previously, SNV N was expressed from pSNV N Tri-x 1.1, generated by cloning the SNV nucleocapsid gene into the NcoI and HindIII sites of pTriEx 1.1 (32). This enables expression of N with a C-terminal his tag in Escherichia coli or HeLa cells. The GFP gene was PCR-amplified from pEGFP plasmid (Clontech) and cloned into pSNVN TriEx 1.1 between NcoI and HindIII sites to generate pT-GFP. T-GFP-N was generated by PCR amplifying the N gene using flanking primers containing EcoRI and NotI sites and cloned into the corresponding sites of pT-GFP.

### Real-Time PCR Analysis.

HeLa cells in six-well plates were co-transfected with a total of 0.4′g of plasmid DNA expressing appropriate RNA as indicated in the text. Appropriate amounts of empty vector were added to the DNA samples to maintain a constant concentration of 0.4′g DNA in each transfection. Each transfection was carried out in triplicate. Thirty-six hours after transfection, cells were lysed and total RNA was isolated using RNeasy (Qiagen), including treatment with RNase-free DNase I (Qiagen), following the manufacturer's protocol. Twenty-five nanograms of total RNA from each well was reverse transcribed using Mo-MLV reverse transcriptase and random primers in a total volume of 50′l. Two microliters of the resulting cDNA were used in 20′l real-time PCR reactions. The relative standard curve method was used for real-time PCR using an ABI prism 7700 sequence detection system following the manufacturer's protocol (Applied Biosystems). Primers targeting the 50 nucleotides on either 5′ or 3′ termini of the mRNA (or nsRNA) of interest and amplification of ′-actin mRNA was used as an “inter control.” The primers used are as follows: 5′ RNA termini from pTriEx, pT-GFP and pT-GFP-ns: GGGAGTCGCTGCGC and GTGAGTCGTATTAATTTCGG; a 3′RNA region from pTriEx: GAAGCUUGCGGCCGCACAGCU and CGATCTCAGTGGTATTTGT; a 3′ RNA region from pT-GFP and pTGFPns: GAAGCUUGCGGCCGCACAGCU and CGATCTCAGTGGTATTTGT; primers for amplification of viral mRNAs containing caps from GFP mRNA and nsRNA: GGGAGTCGCT and GCTCTGTAATGTGCTTTTG; primers for ′-actin: CCATCATGAAGTGTGACGTGG and GTCCGCCTAGAAGCATTTGCG. To assure the amplicon specificity of each primer set, the PCR products were subjected to melting curve analysis followed by sequential agarose gel electrophoresis. The efficiency for amplification of the target (5′ or 3′ mRNA termini) and the internal control gene (′-actin) was examined using serial dilutions of cDNA with gene-specific primers. The mean difference between threshold cycle number values was calculated for each cDNA dilution. The mean difference values corresponding to each dilution were plotted and fit to a straight line with a slope of ′0.1. After this validation test, the levels of stable 5′ and 3′ termini of the test mRNA expressed in HeLa or Vero cells from each of the RNAs was calculated following normalization to the ′-actin mRNA levels and expressed as relative units.

### Confocal Microscopy.

Cells in six-well plates were grown on cover slips and transfected with 0.05°g of pT-GFP-N for the expression of GFP-N fusion peptide.

After 36 h of transfection, cells were fixed with paraformaldehyde-PBS solution for 15 min at room temperature, washed twice with PBS solution, and permeabilized by the addition of 100′l of permeabilization buffer (0.1% triton X-100 in PBS solution) at room temperature for 5 minutes. Cells were washed twice with PBS solution and blocked at room temperature for 30 min by the addition of 100′l of blocking buffer (4% BSA, 1′g goat serum in PBS solution) containing 1′l of goat serum (1′g/′l). Cells were incubated at room temperature for 1 hour with 100′l of primary antibody solution (2′g of anti-Dcp1a monoclonal antibody in 100′l of blocking buffer) and washed three times with PBS solution. Cells were incubated at room temperature for 1 hour with 100′l of rabbit anti-mouse secondary antibody at a 1:100 dilution in blocking buffer, and washed three times with PBS solution. Cover slips were slide mounted using Vectashield plus DAPI (Vector Labs). Microscopy photos were taken on a Zeiss META confocal microscope with a ′63 objective. Images in this paper were generated in the University of New Mexico Cancer Center Fluorescence Microscopy Facility.

### N Pull-Down Assays.

HeLa cells were either mock transfected or transfected with empty vector (pTriEx 1.1) or pT-GFP-N, which contained a C-terminal oligohistidine tag. As a further negative control, cells were transfected with pT-GFP, which also contains a C-terminally his-tagged GFP. After 36 h of transfection, cells were treated with lysis buffer (50 mM Na2PO4, 300 mM NaCl, 10 mM imidazole, pH 8.0) by repetitive passage through a 0.5′16-mm needle and centrifuged, and the transparent lysate was incubated with Ni-NTA beads. Beads were washed three times with wash buffer (50 mM Na2PO4, 300 mM NaCl, 20 mM imidazole, pH 8.0) and bound protein was eluted from beads with elution buffer (50 mM Na2PO4, 300 mM NaCl, 250 mM imidazole, pH 8.0). Eluted materials were analyzed by Western blotting using either anti-DCP1 monoclonal antibody or polyclonal anti-SNV N antibody.

### Co-Immunoprecipitation Assays.

HeLa cells in six-well plates were transfected with the same plasmids and controls indicated earlier. Thirty-six hours after transfection, cells from each well were lysed with 300′l of lysis buffer (50 mM Na2PO4, 300 mM NaCl, pH 8.0) by repetitive passage through a 0.5′16-mm needle and centrifuged, and the transparent lysate containing protease inhibitor (complete mini; Roche Diagnostics) was incubated with l′ g of anti-Dcp1 monoclonal antibody overnight at 4° C. with agitation. Fifty microliters of protein G-coupled Sepharose beads, washed three times with the same lysis buffer, were added to the lysate, followed by further incubation at 4° C. for 4 hours. Beads were pelleted by centrifugation and washed three times with lysis buffer, and 50′l of 2′ SDS gel loading buffer was added. Protein samples were heated in a boiling water bath and loaded directly on SDS gel. Further analysis was carried out by Western blotting using either anti-SNV N or anti-DCP1 antibody. Virus infection and detection of capped viral mRNA. One hundred thousand low passage Vero E6 cells were mock transfected or transfected with pT-GFP or pT-GFPnm. Twenty-four hours after transfection, cells were infected at a multiplicity of infection of 1.5 with Sin Nombre hantavirus SN77734 under BSL3 conditions. Four hours after infection, the cells were rinsed with PBS solution and media were added. Forty-eight hours after infection, RNA was prepared from infected cells. After removal from BSL3, real-time PCR was then carried out using the primers and conditions described in detail in Real-Time PCR.

### Examples 7-10

### Flow Cytometry

All transfections were carried out in triplicate in six-well plates. Flow cytometry was carried out using a FACScan (BD Biosciences), obtaining 10,000 gated events for each sample. The fluorescence of the gated cells was quantified and a histogram was generated to display the distribution of fluorescence intensity in the cell population. The mean fluorescence value of positive and negative events was calculated.

### Ribosome-Loading Assay.

RNA molecules generated were 415 nt long, containing a 200 nt noncoding sequence at the 5′ end, followed by an AUG and 200 additional nucleotides followed by a 15-nt-long poly A tail. Here, 5 mg of this mRNA was added to 20 ml in vitro translation reactions with or without N and incubated at 301 C for 15 min. RNA was recovered with 20 ml of oligotex (poly-dT beads) and reverse transcribed using random primers as described in the ‘Real-time PCR’ section. Then, 2 μl of the resulting cDNA was used for the quantitation of 18S and 28S rRNA using appropriate primers with the standard curve method as in the Real-time PCR section.

### Plasmids.

pSNV N TriEx 1.1 expresses N containing a C-terminal histidine tag both in vivo and in vitro (Mir et al., 2006). Expression and purity of N in E. coli was routinely monitored by gel analysis. pEGF-P (Promega) and pGL3 plasmid (Clone Tech) were used for the expression of Green fluorescence protein (GFP) and luciferase (luc), respectively. pF/HRV-16 2A, which expresses human rhinovirus 162A protease from the EMCV IRES at high level, was kindly provided by Yury Bochkov, Alex Aminev, and Ann Palmenberg (Bochkov and Palmenberg, 2006).
Preparation of mRNA Substrates by In Vitro T7 Transcription Reactions.
We synthesized mRNA molecules for translational expression of GFP, luciferase and N in rabbit reticulocyte lysates, using the Ribomax T7 kit (Promega). Some mRNA molecules contained noncoding 5′ leader sequences 150 nucleotides in length followed by the appropriate coding region and 30 nucleotide long 3′ poly A tail. The SNV N gene was PCR amplified from pGEX-SNV N (Mir and Panganiban, 2004) using two opposing primers. The forward primer was (5′GCTCTAATACGACTCACTATAGGGCCTTTGCAGGGCTGGGAAGC 3′) and the reverse primer was (5′-(T)₃₀GCACAGGAGGGGTAAGCTTTTAAAG 3′). The forward primer contained a proximal T7 RNA polymerase promoter and the reverse primer contained a proximal poly A tail. The PCR product was gel purified and used as a template in T7 transcription reactions. A similar strategy was used for the PCR amplification of the GFP and luciferase genes from pEGFP and pGL3, respectively. All the primers used were complementary to the plasmid sequence outside the gene of interest, so that flanking 5′ and 3′ non-coding sequences were incorporated into the mRNA. T7 transcription reactions were carried out at 37° C. for 3 hours. The DNA template was degraded with DNase I, the mRNA was purified by RNAeasy (Qiagen), and stored in 10 ul aliquots at −70° C. mRNA molecules with terminal 5′ m7G caps were synthesized by the incorporation of m7G cap analog in the transcription reactions following the manufacturers protocol (Promega).

Short RNA molecules, three to six nucleotides long, with or with out a 5′ cap, were synthesized from a 100 nucleotide long DNA template containing a terminal T7 promoter. These short RNA molecules were used for filter binding experiments with SNV N, as described further below. A terminal 5′ cap was incorporated into the short transcripts by adding an m7G cap analog to the T7 reaction mixture. Transcription reactions were depleted of ATP to terminate the reaction after the incorporation of first six nucleotides in the transcript resulting in the synthesis of oligoribonucleotides from three to six nucleotides in length. Reaction mixtures were fractionated on denaturing 18% polyacrylamide gels containing urea. This resulted in a ladder composed of RNAs three, four, five and six nucleotides in length corresponding to (5′-GUC), (5′-GUCU), (5′-GUCUC) and (5′-GUCUCC). Gel slices containing each RNA were excised, crushed, and incubated with 500 ul of probe elution buffer (0.5 M NH₄ acetate, 1 mM EDTA, 0.2% SDS) overnight, followed by centrifugation at 13,000 rpm for 10 minutes. RNA was precipitated from the supernatant by the addition of 0.5M NH₄ acetate and 2.5 volumes of ethanol at −20° C. for 30 minutes. Samples were centrifuged at 13,000 rpm for 30 minutes, the pellet was air dried and dissolved in 100 ul of RNase free water, and stored in 10 ul aliquots at −70° C.

### In Vitro Translation in Rabbit Reticulocyte Lysates.

Nuclease-treated rabbit reticulocyte lysates were used for the translation of mRNA in presence and absence of supplemented, bacterially expressed nucleocapsid protein. Translation reactions were carried out in 50 ul containing 35 ul of rabbit reticulocyte lysate, 1 ul amino acid mixture minus methionine (1 mM), 1 ul S³⁵ methionine (1175 Ci/mmol), 2 ul RNase inhibitor (40 u/ul), 4 ul mRNA in water (250 ng/ul) and 7 ul of RNase free water. The final RNA concentration in the reactions was approximately 90 nM. Reaction mixtures were incubated at 30° C. for 30 minutes. Under these reaction conditions amino acid incorporation continues for about 60 minutes. However, the rate of incorporation appears to decrease over time. Samples were electrophoresed on 10% SDS gels and quantified using a phosphorimager.

### Real Time PCR.

HeLa cells were co-transfected with pEGF-P or pBGL3 and SNV N Trix1.1. After 36 hours, cells from each well were harvested and total RNA was isolated using “RNAeasy” (Qiagen) including treatment with RNase free DNase I (Qiagen). Twenty-five ng of total RNA from each well was reverse transcribed using MMLV reverse transcriptase using random primers in a total volume of 50 ul. Two ul of the resulting cDNA was used in 20 ul real time PCR reactions. An absolute standard curve was used for real time PCR, using a sequence detection system ABI prism 7700, following the manufacturers protocol (Applied Biosystems). A standard curve was generated by amplifying a 150 nucleotide long sequence of GFP or luciferase gene using serially diluted pEGF-P or pBGL3 plasmids as templates. The primers used for GFP amplification were: F primer: 5′-CTGACCTACGGCGTGCAGTGC, and R primer: 5′ CTTCACCTCGGCGGCGGTCTT). Similarly the primers used for the amplification of luciferase were: F primer: 5′ ACGGATTACCAGGGAAAA, and R primer: 5′ GACACCTTTAGGCAGACCAGT. Primer validation was carried out following the manufacturers protocol (Applied Biosystems). Real time PCR reactions were carried out in 20 ul, including 10 ul ribogreen mastermix (Applied Biosystems), 2 ul of template, 3.6 ul of each forward and reverse primer and 0.8 ul water. Reactions were carried out in triplicate.

Detection of the 40S and 60S ribosomal subunits in the pull down experiments with N (on Ni-NTA beads), and with mRNA (on poly-dT beads) was carried out by quantifying 18S and 28S rRNAs using real time PCR. Total RNA from rabbit reticulocyte lysates was purified by RNAeasy kit and reverse transcribed using random primers. A 150 nucleotide long DNA sequence corresponding to 18S ribosomal RNA gene was amplified using two opposing primers (5′ TTATCGGAATTAACCAGAC and 5′ AAAGCTGAAACTTAAAGGAAT) and cloned into a TA tailing vector. A similar strategy was used to amplify and clone a 150 nucleotide sequence corresponding to the 28S rRNA gene using two opposing primers (5′ CCGGATAAAACTGCTTCGGT and 5′ TGGTGAACTATGCCTGGGCAGGGC). The resulting two plasmids, harboring these 18S and 28S gene segments were used for the generation of standard curve in real time PCR analysis. RNA recovered from Ni-NTA or poly-dT beads was purified using RNA easy, and 5 ng of the recovered RNA were reverse transcribed by M-MLV reverse transcriptase using random primers. Two ul of the recovered cDNA were used in real time PCR reactions. We used the absolute standard curve method using cybergreen master mix (Applied Biosystems) for quantification, as described above.

### Luciferase Assays.

HeLa cells were co-transfected with pGL3 (Clonetech) and SNV N Tri-x1.1 plasmids to monitor the effect of N on the luciferase expression. Transfections were carried out in six well plates in a manner similar to that described above for GFP expression. Thirty-six hours after transfection, cells from each well were lysed with 200 ul of lysis buffer, centrifuged at 12,000 g for 2 minutes at 4° C. and supernatant was assayed for the luciferase activity following the manufacturers protocol (Promega).

### RNA Filter Binding.

Interaction of hantavirus N with capped or uncapped GFP mRNA and short RNA molecules three to six nucleotides long was studied by filter binding. RNA molecules were synthesized in vitro with T7 transcription reaction and radiolabeled with P³² CTP during synthesis, as described above. All binding reactions were carried out in RNA binding buffer (Mir and Panganiban, 2004) at a constant concentration of RNA (1 pM) with increasing concentration of N protein. Reaction mixtures were incubated at room temperature for 30-45 minutes and filtered through nitrocellulose membranes under vacuum. Filters were washed with 10 ml of RNA binding buffer and dried. The amount of RNA retained on the filter at different input concentrations of N was measured using a scintillation counter. Data points were fit to a hyperbolic equation using the program Origin 6 (Microcal). The apparent dissociation constant (K_{d}) corresponding to the concentration of N protein required to obtain the half saturation in the binding profile, assuming that the complex formation obeys a simple bimolecular equilibrium. We assumed that the plateau in the binding profile represents complete binding of RNA to allow calculation at half saturation.

“Pull-Down” Experiments with Ni-NTA.

To assess the interaction of N with different components of translational machinery in rabbit reticulocyte lysates we incubated 1.5 ug of SNV N with 10 ul of rabbit reticulocyte lysate in the absence of mRNA, at 30° C. for 30 minutes. Reaction mixtures were loaded on Ni-NTA columns (Qiagen) prewashed with lysis buffer (50 mM NaH2PO4, 300 mM NaCl, 10 mM imidazol) and centrifuged at 700×g for 2 minutes at 4° C. Ni-NTA columns were washed three times with 600 ul of wash buffer (50 mM NaH₂PO₄, 300 mM NaCl, 100 mM imidazol). Bound material was eluted from both the column with 50 ul of elution buffer (50 mM NaH2PO4, 300 mM NaCl, 500 mM imidazol). 25 ul of the eluted sample were used for the purification of total RNA by using RNAeasy. Five ng of the total RNA were reverse transcribed in a 50 ul reaction using random primers as described above. Two ul of the resulting cDNA were used in real time PCR using specific primers for either 18S rRNA or 28S rRNA to check the presence of 40S or 60S ribosomal subunits in the pooled samples. Another 25 ul of the pooled sample were used for the detection of ribosomal proteins or other initiation factors involved in translation by Western blot analysis.

HeLa and 293 cells in six well plates were transfected with SNV N Tri-X 1.1 vector and lysed after 36 hours of transfection. Cells were lysed with 0.2 ml of lysis buffer (50 mM NaH2PO4, 300 mM NaCl, 10 mM imidazol), centrifuged at 10,000 g for 30 min and supernatant was loaded onto Ni-NTA columns, previously washed with lysis buffer. Columns were washed with 600 ul of wash buffer and bound material was eluted with elution buffer as described above. Total RNA purification and Real time PCR studies were carried out as described above. Pooled fractions were also analyzed for the presence of translation initiation factors (eIF4E, eIF4G, unphosphorylated and phosphorylated eIF2a, and ribosomal protein S6 using the corresponding antibodies (Cell Signaling Technology).

### 40S Ribosomal Subunit Preparations.

40S ribosomal subunits were purified from rabbit reticulocyte lysates following standard protocols (Pestova et al., 1996). Briefly, rabbit reticulocyte lysates were diluted ten fold in the presence of 1 mM dithiothreitol, and centrifuged for four hours at 100,000×g using a fixed angle NVT 90 rotor. The resulting pellet was resuspended in 5 ml of buffer A (0.25 M sucrose, 0.05M Tris-HCl (pH 7.5), 1 mM DTT, 6 mM MgCl2, and 0.1 mM EDTA), followed by the addition of 0.5 M KCl with continuous stirring on ice for 30 minutes. The mixture was centrifuged for 2 hours at 180,000 g using a NVT 90 rotor. The pellet was dissolved in small volume of buffer A, layered over a sucrose cushion (1.0 M sucrose, 0.5 M KCL, 0.02 M Tris-HCL (pH 7.5), 2 mM MgCl2 and 0.1 mM EDTA) and centrifuged at 275,000 g for 3 hours. The pellet, containing whole 80S ribosomes, was resuspended in a small volume of buffer A and further diluted ten fold in buffer B (0.5 M KCl, 0.05 M Hepes (pH 7.5), 2 mM MgCl2, and 1 mM puromycin). The mixture was incubated for 10 minutes on ice followed by further incubation for 10 minutes at 37° C. and 5 minutes on ice. This limited exposure with puromycin dissociates intact 80S ribosomes into large and small ribosomal subunits. The solution was layered onto a 5-20% sucrose gradient prepared in buffer C (0.5M KCl, 0.05M Hepes (pH 7.5), 5 mM MgCl2, 1 mM DTT and 0.1 mM EDTA) and centrifuged at 50,000 rpm for 3 hours. 0.25 ml fractions were collected and monitored by checking their absorbance at 280 nm. Two peaks corresponding to 40S and 60S subunits were detected. The fractions containing 40S subunit were pooled and concentrated by further centrifugation at 70,000 rpm for 10 hours. The purified 40S subunit pellet was resuspended in subunit storage buffer (0.05M Tris-HCl (pH 7.5), 0.25 M sucrose, 1 mM DTT, 0.1 mM EDTA, 10 mM KCl and 1 mM MgCl2) and stored at −80° C.

Interaction of 40S Subunit with SNV N Protein.

pTri.Ex SNV N (Mir and Panganiban, 2006) was linearized by HindIII and used as template in in vitro T7 transcription system to generate mRNA expressing SNV N with C-terminal octahistidine tag. This mRNA was translated in rabbit reticulocyte lysates and labeled with ³⁵S-met. Labeled N was purified under denaturing conditions from rabbit reticulocyte lysates using a Ni-NTA affinity column. Purified N was renatured, concentrated, and sedimented on a 10-30% sucrose gradient prepared in gradient buffer (0.05M Hepes (pH 7.5), 1 mM DTT, 0.1 mM EDTA, 10 mM KCl and 1 mM MgCl2). The peak of N was in fraction 16 (FIG. 7B). In parallel, N was incubated with purified 40S ribosomal subunits at 37° C. for 1 hour prior to its fractionation on 10-30% sucrose gradient.

Expression and Purification of Wild Type and Mutant eIF4AI.

Wild type eIF4AI was expressed from Pet 36-4AI and mutant eIF4AI was expressed from Pet 36-R362Q (Pause et al., 1994). These two expression vectors were generously provided by Nahum Sonenberg and Colin Lister. BL21 cells transformed with the expression constructs were inoculated into one liter of LB media and allowed to grow at 37° C. for 5 hrs to an OD₅₉₅ of 1.0. Cells were induced with IPTG and allowed to grow for an additional 3 hours and centrifuged at 3000 rpm for 30 minutes. The bacterial pellet was suspended in 20 ml of buffer A (20 mM Tris, pH 7.5, 10% glycerol, 0.1 mM EDTA and 2 mM DTT). The suspension was sonicated eight times with fifteen second bursts and centrifuged at 20,000×g for 20 minutes. The supernatant was incubated with 40% ammonium sulfate with continuous stirring at 4° C., followed by centrifugation at 10,000×g for 10 minutes. The pellet was discarded and the supernatant incubated with 80% ammonium sulfate with continuous stirring, followed by centrifugation as above. The supernatant was discarded and the pellet was resuspended in two volumes of buffer A and dialyzed overnight. After dialysis, material was loaded into 5 ml DEAE sephacel column and washed with buffer A, containing 100 mM KCl. A KCl gradient (0.1M to 0.5 M KCl) was applied in buffer A. eIF4A elutes from the column at about 0.2 M KCl. The presence of eIF4A in gradient fractions was confirmed by Western analysis using anti eIF4A antibodies (provided by the Sonenberg lab). Fractions were pooled and diluted with buffer A (1:1 dilution). Diluted fractions were loaded onto Hi-Trap blue columns (Pharmacia), followed by washing with buffer A containing 100 mM KCl. A KCl gradient (0.1M-2M KCl) in buffer A was applied. eIF4A elutes from the column at about 1M KCl. Fractions containing eIF4A were dialyzed with buffer A containing 100 mM KCl and loaded onto a mono Q 5/5 Column and washed with buffer A containing 120 mM KCl. Sequential KCl gradients (120-160 mM KCl) in 10 ml of buffer A, (160-200 mM KCl) in 20 ml, and (200-240 mM KCl) in 10 ml were applied to the column. eIF4A elutes from the column between 180-190 mM KCl. Fractions containing eIF4A were pooled, concentrated and used in translation experiments.

### Examples 7-10 Materials and Methods Citations

Bochkov, Y. A., and Palmenberg, A. C. (2006). Translational efficiency of EMCV IRES in bicistronic vectors is dependent upon IRES sequence and gene location. Biotechniques 41, 283-284, 286, 288 passim.
Mir, M. A., Brown, B., Hjelle, B., Duran, W. A., and Panganiban, A. T. (2006). Hantavirus N protein exhibits genus-specific recognition of the viral RNA panhandle. J Virol 80, 11283-11292.
Mir, M. A., and Panganiban, A. T. (2004). Trimeric hantavirus nucleocapsid protein binds specifically to the viral RNA panhandle. J Virol 78, 8281-8288.
Mir, M. A., and Panganiban, A. T. (2006). The bunyavirus nucleocapsid protein is an RNA chaperone: possible roles in viral RNA panhandle formation and genome replication. Rna 12, 272-282.
Pause, A., Methot, N., Svitkin, Y., Merrick, W. C., and Sonenberg, N. (1994). Dominant negative mutants of mammalian translation initiation factor eIF-4A define a critical role for eIF-4F in cap-dependent and cap-independent initiation of translation. Embo J 13, 1205-1215.
Pestova, T. V., Hellen, C. U., and Shatsky, I. N. (1996). Canonical eukaryotic initiation factors determine initiation of translation by internal ribosomal entry. Mol Cell Biol 16, 6859-6869.

### REFERENCES

### First Set

Ali I K, McKendrick L, Morley S J, Jackson R T (2001) Truncated initiation factor eIF4G lacking an eIF4E binding site can support capped mRNA translation. EMBO J. 20: 4233-4242
Barr J N (2007) Bunyavirus mRNA synthesis is coupled to translation to prevent premature transcription termination. RNA 13: 731-736
Bellocq C, Kolakofsky D (1987) Translational requirement for La Crosse virus S-mRNA synthesis: a possible mechanism. J Virol 61: 3960-3967
Blakqori G, Kochs G, Haller O, Weber F (2003) Functional L polymerase of La Crosse virus allows in vivo reconstitution of recombinant nucleocapsids. J Gen Virol 84 (Part 5): 1207-1214
Bochkov Y A, Palmenberg A C (2006) Translational efficiency of EMCV IRES in bicistronic vectors is dependent upon IRES sequence and gene location. Biotechniques 41: 283-284, 286, 288 passim
Bol J F (2005) Replication of alfamo- and ilarviruses: role of the coat protein. Annu Rev Phytopathol 43: 39-62
Boudko S P, Kuhn R J, Rossmann M G (2007) The coiled-coil domain structure of the Sin Nombre virus nucleocapsid protein. J Mol Biol 366: 1538-1544
Bridgen A, Elliott R M (1996) Rescue of a segmented negative-strand RNA virus entirely from cloned complementary DNAs. Proc Natl Acad Sci USA 93: 15400-15404
Burgui I, Yanguez E, Sonenberg N, Nieto A (2007) Influenza virus mRNA translation revisited: is the eIF4E cap-binding factor required for viral mRNA translation? J Virol 81: 12427-12438
Castello A, Sanz M A, Molina S, Carrasco L (2006) Translation of Sindbis virus 26S mRNA does not require intact eukaryotic initiation factor 4G. J Mol Biol 355: 942-956
Connor J H, Lyles D S (2002) Vesicular stomatitis virus infection alters the eIF4F translation initiation complex and causes dephosphorylation of the eIF4E binding protein 4E-BP1. J Virol 76:10177-10187
Dever T E (1999) Translation initiation: adept at adapting. Trends Biochem Sci 24: 398-403
Dunn E F, Pritlove D C, Jin H, Elliott R M (1995) Transcription of a recombinant bunyavirus RNA template by transiently expressed bunyavirus proteins. Virology 211: 133-143
Etchison D, Milburn S C, Edery I, Sonenberg N, Hershey J W (1982) Inhibition of HeLa cell protein synthesis following poliovirus infection correlates with the proteolysis of a 220,000-dalton polypeptide associated with eucaryotic initiation factor 3 and a cap binding protein complex. J Biol Chem 257: 14806-14810
Garcin D, Lezzi M, Dobbs M, Elliott R M, Schmaljohn C, Kang C Y, Kolakofsky D (1995) The 5′ ends of Hantaan virus (Bunyaviridae) RNAs suggest'a prime-and-realign mechanism for the initiation of RNA synthesis. J Virol 69: 5754-5762
Gingras A C, Raught B, Sonenberg N (1999) eIF4 initiation factors: effectors of mRNA recruitment to ribosomes and regulators of translation. Annu Rev Biochem 68: 913-963
Gott P, Stohwasser R, Schnitzler P, Darai G, Bautz E K (1993) RNA binding of recombinant nucleocapsid proteins of hantaviruses. Virology 194: 332-337
Gray N K, Coller J M, Dickson K S, Wickens M (2000) Multiple portions of poly(A)-binding protein stimulate translation in vivo. EMBO J. 19: 4723-4733
Haghighat A, Svitkin Y, Novoa I, Kuechler E, Skern T, Sonenberg N (1996) The eIF4G-eIF4E complex is the target for direct cleavage by the rhinovirus 2A proteinase. J Virol 70: 8444-8451.
Hellen C U, Sarnow P (2001) Internal ribosome entry sites in eukaryotic mRNA molecules. Genes Dev 15: 1593-1612
Hentze M W (1997) eIF4G: a multipurpose ribosome adapter? Science 275: 5′0-5′1
Hernandez G, Vazquez-Pianzola P (2005) Functional diversity of the eukaryotic translation initiation factors belonging to eIF4 families. Mech Dev 122: 865-876
Hershey J W B, Merrick W C (2000) Pathway and mechanism of initiation of protein synthesis. In Translational Control of Gene Expression, Sonenberg N, Hershey J W B, Mathews M B (eds), pp 33-88. Cold Spring Harbor, N.Y.: Cold Spring Harobor Press
Hinnebusch A G (2006) eIF3: a versatile scaffold for translation initiation complexes. Trends Biochem Sci 31: 553-562
Hutchinson K L, Peters C J, Nichol S T (1996) Sin Nombre virus mRNA synthesis. Virology 224: 139-149
Ikegami T, Peters C J, Makino S (2005) Rift valley fever virus nonstructural protein NSs promotes viral RNA replication and transcription in a minigenome system. J Virol 79: 5606-5615
Jang S K (2006) Internal initiation: IRES elements of picornaviruses and hepatitis C virus. Virus Res 119: 2-15
Jonsson C B, Gallegos J, Ferro P, Severson W, Xu X, Schmaljohn C S, Fero P (2001) Purification and characterization of the Sin Nombre virus nucleocapsid protein expressed in Escherichia coli. Protein Expr Purif 23: 134-141
Jonsson C B, Schmaljohn C S (2001) Replication of hantaviruses. Curr Top Microbiol Immunol 256: 15-32
Kang B C, Yeam I, Frantz J D, Murphy J F, Jahn M M (2005) The pvr1 locus in capsicum encodes a translation initiation factor eIF4E that interacts with tobacco etch virus VPg. Plant J 42: 392-405
Kohl A, Hart T J, Noonan C, Royall E, Roberts L O, Elliott R M (2004) A bunyamwera virus minireplicon system in mosquito cells. J Virol 78: 5679-5685
Korneeva N L, Lamphear B J, Hennigan F L, Rhoads R E (2000) Mutually cooperative binding of eukaryotic translation initiation factor (eIF) 3 and eIF4A to human eIF4G-1. J Biol Chem 275: 41369-41376
Kozak M (1991) Structural features in eukaryotic mRNAs that modulate the initiation of translation. J Biol Chem 266:19867-19870
Kozak M (1992) Regulation of translation in eukaryotic systems. Annu Rev Cell Biol 8: 197-225
Liebig H D, Ziegler E, Yan R, Hartmuth K, Klump H, Kowalski H, Blaas D, Sommergruber W, Frasel L, Lamphear B (1993) Purification of two picornaviral 2A proteinases: interaction with eIF-4 gamma and influence on in vitro translation. Biochemistry 32: 7581-7588
Mader S, Lee H, Pause A, Sonenberg N (1995) The translation initiation factor eIF-4E binds to a common motif shared by the translation factor eIF-4 gamma and the translational repressors 4E-binding proteins. Mol Cell Biol 15: 4990-4997
Marcotrigiano J, Gingras A C, Sonenberg N, Burley S K (1997) Cocrystal structure of the messenger RNA 5′ cap-binding protein (eIF4E) bound to 7-methyl-GDP. Cell 89: 951-961
Marcotrigiano J, Lomakin I B, Sonenberg N, Pestova T V, Hellen C U, Burley S K (2001) A conserved HEAT domain within eIF4G directs assembly of the translation initiation machinery. Mol Cell 7: 193-203
Matsuo H, Li H, McGuire A M, Fletcher C M, Gingras A C, Sonenberg N, Wagner G (1997) Structure of translation factor eIF4E bound to m7GDP and interaction with 4E-binding protein. Nat Struct Biol 4: 717-724
Mir M A, Brown B, Hjelle B, Duran W A, Panganiban A T (2006) Hantavirus N protein exhibits genus-specific recognition of the viral RNA panhandle. J Virol 80: 11283-11292
Mir M A, Panganiban A T (2004) Trimeric hantavirus nucleocapsid protein binds specifically to the viral RNA panhandle. J Virol 78: 8281-8288
Mir M A, Panganiban A T (2005) The hantavirus nucleocapsid protein recognizes specific features of the viral RNA panhandle and is altered in conformation upon RNA binding. J Virol 79:1824-1835
Mir M A, Panganiban A T (2006) The bunyavirus nucleocapsid protein is an RNA chaperone: possible roles in viral RNA panhandle formation and genome replication. RNA 12: 272-282
Obijeski J F, Bishop D H, Murphy F A, Palmer E L (1976) Structural proteins of La Crosse virus. J Virol 19: 985-997
Osborne J C, Elliott R M (2000) RNA binding properties of bunyamwera virus nucleocapsid protein and selective binding to an element in the 5′ terminus of the negative-sense S segment. J Virol 74: 9946-9952
Pause A, Methot N, Svitkin Y, Merrick W C, Sonenberg N (1994) Dominant negative mutants of mammalian translation initiation factor eIF-4A define a critical role for eIF-4F in capdependent and cap-independent initiation of translation. EMBO J. 13: 1205-1215
Pestova T V, Hellen C U, Shatsky I N (1996) Canonical eukaryotic initiation factors determine initiation of translation by internal ribosomal entry. Mol Cell Biol 16: 6859-6869
Pettersson R F, von Bonsdorff C H (1975) Ribonucleoproteins of Uukuniemi virus are circular. J Virol 15: 386-392 N replaces eIF4F
M A Mir, A T Panganiban Pinschewer D D, Perez M, de la Torre J C (2003) Role of the virus nucleoprotein in the regulation of lymphocytic choriomeningitis virus transcription and RNA replication. J Virol 77:3882-3887
Piron M, Vende P, Cohen J, Poncet D (1998) Rotavirus RNA-binding protein NSP3 interacts with eIF4GI and evicts the poly(A) binding protein from eIF4F. EMBO J. 17: 5811-5821
Raju R, Kolakofsky D (1989) The ends of La Crosse virus genome and antigenome RNAs within nucleocapsids are base paired. J Virol 63: 122-128
Richter J D, Sonenberg N (2005) Regulation of cap-dependent translation by eIF4E inhibitory proteins. Nature 433: 477-480
Rogers Jr G W, Komar A A, Merrick W C (2002) eIF4A: the godfather of the DEAD box helicases. Prog Nucleic Acid Res Mol Biol 72: 307-331
Schmaljohn C M (1996) Molecular Biology of Hantaviruses. New York: Plenum Press
Schutz P, Bumann M, Oberholzer A E, Bieniossek C, Trachsel H, Altmann M, Baumann U (2008) Crystal structure of the yeast eIF4A-eIF4G complex: an RNA-helicase controlled by protein-protein interactions. Proc Natl Acad Sci USA 105: 9564-9569
Severson W, Partin L, Schmaljohn C S, Jonsson C B (1999) Characterization of the Hantaan nucleocapsid protein-ribonucleic acid interaction. J Biol Chem 274: 33732-33739
Severson W E, Xu X, Jonsson C B (2001) cis-Acting signals in encapsidation of Hantaan virus S-segment viral genomic RNA by its N protein. J Virol 75: 2646-2652
Shirokikh N E, Spirin A S (2008) Poly(A) leader of eukaryotic mRNA bypasses the dependence of translation on initiation factors. Proc Natl Acad Sci USA 105: 10738-10743
Svitkin Y V, Ovchinnikov L P, Dreyfuss G, Sonenberg N (1996) General RNA binding proteins render translation cap dependent. EMBO J. 15: 7147-7155
Tarun Jr S Z, Sachs A B (1996) Association of the yeast poly(A) tail binding protein with translation initiation factor eIF-4G. EMBO J. 15: 7168-7177
von der Haar T, Gross J D, Wagner G, McCarthy J E (2004) The mRNA cap-binding protein eIF4E in post-transcriptional gene expression. Nat Struct Mol Biol 11: 5′3-511
Wiegand M A, Bossow S, Schlecht S, Neubert W J (2007) De novo synthesis of N and P proteins as a key step in Sendai virus gene expression. J Virol 81: 13835-13844.

### REFERENCES

### Second Set

1. Li M L, Ramirez B C, Krug R M (1998) RNA-dependent activation of primer RNA production by influenza virus polymerase: different regions of the same protein subunit constitute the two required RNA-binding sites. EMBO J. 17:5844-5852.
2. Hagen M, Chung T D, Butcher J A, Krystal M (1994) Recombinant influenza virus polymerase: requirement of both 5′ and 3′ viral ends for endonuclease activity. J Virol 68:15′9-1515.
3. Duijsings D, Kormelink R, Goldbach R (2001) In vivo analysis of the TSWV cap-snatching mechanism: single base complementarity and primer length requirements. EMBO J. 20:2545-2552.
4. Estabrook E M, Tsai J, Falk B W (1998) In vivo transfer of barley stripe mosaic hordeivirus ribonucleotides to the 5′ terminus of maize stripe tenuivirus RNAs. Proc Natl Acad Sci USA 95:8304-8309.
5. Ramirez B C, Garcin D, Calvert L A, Kolakofsky D, Haenni A L (1995) Capped nonviral sequences at the 5′ end of the mRNAs of rice hoja blanca virus RNA4. J Virol 69:1951-1954.
6. Vialat P, Bouloy M (1992) Germiston virus transcriptase requires active 40S ribosomal subunits and utilizes capped cellular RNAs. J Virol 66:685-693.
7. Jin H, Elliott R M (1993) Characterization of Bunyamwera virus S RNA that is transcribed and replicated by the L protein expressed from recombinant vaccinia virus. J Virol 67:1396-1404.
8. Garcin D, et al. (1995) The 5′ ends of Hantaan virus (Bunyaviridae)) RNAs suggest a prime-and-realign mechanism for the initiation of RNA synthesis. J Virol 69:5754-5762.
9. Meyer S, Temme C, Wahle E (2004) Messenger RNA turnover in eukaryotes: pathways and enzymes. Crit. Rev Biochem Mol Biol 39:197-216.
10. Parker R, Sheth U (2007) P bodies and the control of mRNA translation and degradation. Mol Cell 25:635-646.
11. Eulalio A, Behm-Ansmant I, Izaurralde E (2007) P bodies: at the crossroads of posttranscriptional pathways. Nat Rev Mol Cell Biol 8:9-22.
12. Brengues M, Teixeira D, Parker R (2005) Movement of eukaryotic mRNAs between polysomes and cytoplasmic processing bodies. Science 310:486-489.
13. Bhattacharyya S N, Habermacher R, Martine U, Closs E I, Filipowicz W (2006) Relief of microRNA-mediated translational repression in human cells subjected to stress. Cell 125:1111-1124.
14. Stalder L, Muhlemann O (2008) The meaning of nonsense. Trends Cell Biol 18:315-321.
15. Conti E, Izaurralde E (2005) Nonsense-mediated mRNA decay: molecular insights and mechanistic variations across species. Curr Opin Cell Biol 17:316-325.
16. Lejeune F, Maquat L E (2005) Mechanistic links between nonsense-mediated mRNA decay and pre-mRNA splicing in mammalian cells. Curr Opin Cell Biol 17:309-315.
17. Amrani N, Sachs M S, Jacobson A (2006) Early nonsense: mRNA decay solves a translational problem. Nat Rev Mol Cell Biol 7:415-425.
18. Le Hir H, Seraphin B (2008) EJCs at the heart of translational control. Cell 133:213-216.
19. van Dijk E, et al. (2002) Human Dcp2: a catalytically active mRNA decapping enzyme located in specific cytoplasmic structures. EMBO J. 21:6915-6924.
20. Ingelfinger D, Arndt-Jovin D J, Luhrmann R, Achsel T (2002) The human LSm1-7 proteins colocalize with the mRNA-degrading enzymes Dcp1/2 and Xrnl in distinct cytoplasmic foci. RNA 8:1489-15′1.
21. Lykke-Andersen J (2002) Identification of a human decapping complex associated with hUpf proteins in nonsense-mediated decay. Mol Cell Biol 22:8114-8121.
22. Teixeira D, Sheth U, Valencia-Sanchez M A, Brengues M, Parker R (2005) Processing bodies require RNA for assembly and contain nontranslating mRNAs. RNA 11:371-382:
23. Tarun S Z Jr, Sachs A B (1996) Association of the yeast poly(A) tail binding protein with translation initiation factor eIF-4G. EMBO J. 15:7168-7177.
24. Gray N K, Coller J M, Dickson K S, Wickens M (2000) Multiple portions of poly(A)-binding protein stimulate translation in vivo. EMBO J. 19:4723-4733.
25. Patterson J L, Holloway B, Kolakofsky D (1984) La Crosse virions contain a primer stimulated RNA polymerase and a methylated cap-dependent endonuclease. J Virol 52:215-222.
26. Fechter P, Brownlee G G (2005) Recognition of mRNA cap structures by viral and cellular proteins. J Gen Virol 86:1239-1249.
27. Ramanathan H N, Jonsson C B (2008) New and Old World hantaviruses differentially utilize host cytoskeletal components during their life cycles. Virology 374:138-15′.
28. Bellocq C, Kolakofsky D (1987) Translational requirement for La Crosse virus S-mRNA synthesis: a possible mechanism. J Virol 61:3960-3967.
29. Beliakova-Bethell N, et al. (2006) Virus-like particles of the Ty3 retrotransposon assemble in association with P-body components. RNA 12:94-101.
30. Beckham C J, et al. (2007) Interactions between bromemosaic virus RNAs and cytoplasmic processing bodies. J Virol 81:9759-9768.
31. Mir M A, Panganiban A T (2004) Trimeric hantavirus nucleocapsid protein binds specifically to the viral RNA panhandle. J Virol 78:8281-8288.
32. Mir M A, Brown B, Hjelle B, Duran W A, Panganiban A T (2006) Hantavirus N protein exhibits genus-specific recognition of the viralRNApanhandle. J Virol 80:11283-11292.
33. Botten J, et al. (2000) Experimental infection model for Sin Nombre hantavirus in the deer mouse (Peromyscus maniculatus). Proc Natl Acad Sci USA 97:10578-10583.
34. Mir M A, Panganiban A T (2008) A protein that replaces the entire eIF-4F complex. EMBO J, in press.

1. A method for increasing the production of a gene product from a production cell comprising delivering to said production cell a nucleotide construct which comprises a 5′ untranslated region (UTR) from a virus of the family Bunyaviridae which serves as a translational promoter for high level translation of said gene product to which a translational activator (enhancer) comprising a Bunyaviridae nucleocapsid or active polypeptide portion thereof can bind, a nucleotide region which expresses said gene product, and a start codon comprising a nucleotide sequence, which permits translation of the gene product.

2. The method according to claim 2 wherein a polynucleotide encoding said nucleocapsid is incorporated into said nucleotide construct.

3. The method according to claim 1 wherein said nucleocapsid is from a virus from the genus hantavirus, orthobunyavirus, nairovirus, tospovirus or phlebovirus.

4. The method according to claim 1 wherein said nucleotide construct comprises a polynucleotide according to the structure:
m7G(X)₀₋₁₅(UAG)₂₋₅(X)₀₋₄₀(start codon nucleotides)₅₋₆AUG−polynucleotide encoding a gene product
where m7G represents a cap of the promoter;
X=any nucleotide (including 2′-deoxynucleotides) containing a base selected from the group consisting of guanine, adenine, cytosine, uracil and thymine;
UAG is one copy of a triplet repeat;
start codon nucleotides is any combination of 5 or 6 nucleotides that permit translation of the gene at the 5′AUG nucleotide triplet of the gene; and AUG=beginning (5′ end) of the gene to be translated into protein.

5. The method according to claim 1 wherein said nucleocapsid is a hantavirus nucleocapsid.

6. The method according to claim 3 wherein said nucleocapsid comprises SEQ ID NO: 1.

7. The method according to claim 3 wherein said nucleocapsid comprises SEQ ID NO: 2 linked to a trimerization peptide.

8. The method according to claim 1 wherein said gene product is a protein or polypeptide.

9. The method according to claim 1 wherein said gene product is an antibody, bioactive agent, a drug, a food protein or food additive.

10. A method for increasing the production of a gene product from a production cell comprising delivering to said production cell a nucleotide construct which comprises a nucleotide sequence which encodes a promoter operably linked to a nucleotide sequence which encodes a translational activator comprising a Bunyaviridae nucleocapsid or active polypeptide portion thereof, and a nucleotide region which expresses said gene product.

11. A nucleotide construct comprising a 5′ untranslated region (UTR) from a virus of the family Bunyaviridae which serves as a translational promoter for high level translation of a gene product and which binds to a Bunyaviridae nucleocapsid or active polypeptide portion thereof, a nucleotide region which expresses a gene product protein or polypeptide, and a start codon comprising a nucleotide sequence which permits translation of the gene product.

12. The construct according to claim 11 further comprising a spacer nucleotide group between the translational promoter and the start codon of about 0 to 40 nucleotide units (mer).

13. The construct according to either of claim 11 wherein said nucleocapsid is from a virus from the genus hantavirus, orthobunyavirus, nairovirus, tospovirus or phlebovirus.

14. The construct according to claim 11 wherein a polynucleotide encoding said nucleocapsid is incorporated into said nucleotide construct.

15. The construct according to claim 11 comprising a polynucleotide according to the structure:
m7G(X)₀₋₁₅(UAG)₂₋₅(X)₀₋₄₀(start codon nucleotides)₅₋₆AUG−polynucleotide encoding a gene product
where m7G represents a cap of the promoter;
X=any nucleotide (including 2′-deoxynucleotides) containing a base selected from the group consisting of guanine, adenine, cytosine, uracil and thymine;
UAG is one copy of a triplet repeat;
start codon nucleotides is any combination of 5 or 6 nucleotides that permit translation of the gene at the 5′AUG nucleotide triplet of the gene; and
AUG is the beginning (5′ end) of the gene to be translated into protein.

16. The construct according to claim 11 wherein said nucleocapsid is a hantavirus nucleocapsid.

17. The construct according to claim 11 wherein said nucleocapsid comprises SEQ ID NO: 1.

18. The construct according to claim 11 wherein said nucleocapsid comprises SEQ ID NO: 2 linked to a trimerization peptide.

19. The construct according to claim 11 wherein said gene product is a protein or polypeptide.

20. The construct according to claim 11 wherein said gene product is an antibody, a drug, a food protein or food additive.

21. An isolated cell containing a nucleic acid molecule comprising a gene expression control region which comprises a nucleotide sequence which encodes a promoter operably linked to a nucleotide sequence which encodes a translational activator comprising a Bunyaviridae nucleocapsid or active polypeptide portion thereof, and a nucleotide region which expresses a gene product.

22. The isolated cell of claim 21, wherein translational activator has an amino acid sequence which is at least 60%, or at least 70%, or at least 80%, or at least 80%, or at least 95% identical to either SEQ ID NO: 1 or SEQ ID NO: 2.

23. A method for producing a gene product comprising:
(a) culturing a cell containing a nucleotide construct which comprises a nucleotide sequence which encodes a promoter operably linked to a nucleotide sequence which encodes a translational activator comprising a Bunyaviridae nucleocapsid or active polypeptide portion thereof, and a nucleotide region which expresses said gene product; and
(b) isolating the gene product.

24. The method of claim 23, wherein the gene product is a mammalian protein.

25. The method of claim 23, wherein the gene product is isolated in soluble form.

26. An expression vector comprising a nucleotide sequence which encodes a promoter operably linked to a nucleotide sequence which encodes a translational activator comprising a Bunyaviridae nucleocapsid or active polypeptide portion thereof, and a nucleotide region which expresses a gene product.

27. The expression vector of claim 26, wherein the expression vector comprises a 5′ untranslated region (UTR) from a virus of the family Bunyaviridae which serves as a translational promoter for high level translation of a gene product and which binds to a Bunyaviridae nucleocapsid or active polypeptide portion thereof, a nucleotide region which expresses a gene product protein or polypeptide, and a start codon comprising a nucleotide sequence which permits translation of the gene product

28. A kit which comprises at least one DNA construct of claim 11.
